(19) United States
(12) Patent Application Publication
(10) Pub. No.: US 2006/0094676 A1
(43) Pub. Date: May 4, 2006

(54) Compositions and methods for treating cancer using compositions comprising an inhibitor of endothelin receptor activity

(76) Inventors: Ronit Lahav, Pully (CH); Paul H. Patterson, Altadena, CA (US)

Correspondence Address: DLA PIPER RUDNICK GRAY CARY US, LLP, 4365 EXECUTIVE DRIVE, SUITE 1100, SAN DIEGO, CA, 92121-2133, US

(21) Appl. No.: 10/977,376

(22) Filed: Oct. 29, 2004

(51) Int. Cl. A61K 48/00; A61K 31/517; A61K 31/473; A61K 31/4709; A61K 31/455; A61K 31/165

(52) U.S. Cl. 514/44; 514/248; 514/266.3; 514/309; 514/298; 514/355; 514/620

(57) ABSTRACT

Elevated ETRB activity, BCL-2A1 activity and/or PARP-3 activity was detected in cancer cells, and determined to be associated with growth and proliferation of the cancer cells. Accordingly, methods are provided for treating cancer by reducing or inhibiting the ETRB activity, BCL-2A1 activity and/or PARP-3 activity. Also provided are methods of determining the responsiveness of cancer cells to treatment with inhibitors of ETRB activity, BCL-2A1 activity and/or PARP-3 activity. Further, decreased cell viability was observed to correlate with reduction in ETRB expression, and reduction in ETRB protein levels by siRNA led to an increase in cell death.

### FIELD OF THE INVENTION

The present invention relates generally to the treatment of cancer and more specifically to the use of inhibitors of endothelin receptor activity to reduce cancer cell growth.

### BACKGROUND INFORMATION

One-third of all individuals in the United States will develop cancer. Although the five year survival rate has risen dramatically as a result of progress in early diagnosis and therapy, cancer still remains second only to cardiac disease as a cause of death in the United States. Twenty percent of Americans die from cancer, half due to lung, breast, and colon-rectal cancer. Additionally, skin cancer remains a serious health hazard.

Cancer progression is often associated with reactivation of developmental programs. Consistent with this notion, melanoma cells display a highly proliferative and motile phenotype that is shared with embryonic melanocyte precursors which typically migrate over long distances within the organism. Studies on the mechanisms that regulate melanocyte migration have provided insight into the function of endothelins (ET) and their receptors. The ET family of molecules is composed of three polypeptides, ET-1, ET-2 and ET-3 of 21 amino acids each that bind to two highly homologous G-coupled protein receptors, endothelin receptor A (ETRA) and endothelin receptor B (ETRB), which trigger a variety of signals according to the cell type. ETRB promotes migration and proliferation of early melanocyte precursors, and mutation in ETRB in both humans and mice results in spotting due to the inability of an elevated proportion of melanocytes to reach the skin.

Recently, ETRB was shown to mediate molecular events characteristic of melanoma progression. In support of this view, one of the ETRB ligands, ET-1 is reported to be secreted by skin keratinocytes in response to ultraviolet irradiation, a major triggering factor in melanoma development. Moreover, UV-mediated induction of ET-1 down-regulates E-cadherin in melanocytes and melanoma cells through ETRB. Down-regulation of E-cadherin expression is commonly observed in melanomas and is proposed to enhance their invasiveness. Taken together, these observations suggest that ETRB activation contributes to melanoma development and progression, and inhibition thereof provides a possible mechanism of treating cancer.

Of further interest are the roles played by BCL-2A1 and PARP-3 in cancer progression. BCL-2A1, a bcl-2 family member, has been identified as a hematopoietic-specific, early inducible gene. It has been shown that Bcl-2 transfected B cells were resistant towards apoptosis normally induced in B cells by IL-3 withdrawal. Thus, it was demonstrated that the pathway toward tumorigenesis depends not only on the ability to escape growth control but also depends on the ability to prevent apoptosis. PARP-3, a member of the poly(ADP-ribose) polymerase (PARP) family, has been identified as a core component of the centrosome, and is involved in DNA repair and cell death induction upon DNA damages. PARP cleavage, leading to its inactivation and thereby preventing DNA repair, and thus improving endonuclease access to chromatin, is an early event in apoptosis. These observations suggest that a reduction in BCL-2A1 and PARP-3 expression is implicated in ETRB blockade-dependent cell death.

The ability to modulate one or more genes involved with cancer progression thus represents a possible therapeutic approach to several clinically significant cancers. A need therefore exists for methods and compounds that inhibit endothelin receptor B activity, BCL-2A1 activity and/or PARP-3 activity to treat cancer.

### SUMMARY OF THE INVENTION

The present invention is based, in part, on the determination that ETRB activity is elevated in cancer cells as compared to corresponding normal cells of the subject having the cancer, and that agents that decrease ETRB activity inhibit proliferation of cancer cells. Likewise, the invention is based on the determination that BCL-2A1 activity and PARP-3 activity are elevated in cancer cells as compared to corresponding normal cells of the subject having the cancer, and that agents that decrease BCL-2A1 activity and/or PARP-3 activity inhibit proliferation of cancer cells. Also associated with elevated ETRB activity is decreased HIF-1α activity, decreased VEGF activity and/or increased GRAVIN activity. Thus, it will be recognized that increased HIF-1α activity, increased VEGF activity, and/or decreased GRAVIN activity are useful indicators of decreased ETRB activity.

Accordingly, the present invention provides methods of treating cancer characterized by elevated ETRB activity, BCL-2A1 activity, and/or PARP-3 activity, as well as methods of determining whether cancer cells have such activities, and methods of identifying agents useful for treating such cancers. As such, methods of personalized medicine are provided, wherein agents can be selected that are particularly useful for treating a particular cancer in a subject. Further, methods of monitoring a therapeutic regimen for treating a subject having cancer are provided.

In one embodiment, the method for treating cancer involves administering to a subject in need of treatment for cancer, a therapeutically effective amount of a nucleic acid molecule that results in silencing endothelin receptor B activity through RNAi in cancer cells of the subject. In another embodiment, the method involves administering a therapeutically effective amount of a selective inhibitor of BCL-2A1 activity. In yet another embodiment, the method involves administering a therapeutically effective amount of a selective inhibitor of PARP-3 activity. In yet another embodiment, the method involves administering a therapeutically effective amount of a selective inhibitor of ETRB activity in combination with a therapeutic agent. Methods for monitoring a therapeutic regimen for treating a subject having cancer with such treatments involve determining a change in BCL-2A1 activity, PARP-3 activity, HIF-1α activity, VEGF activity, and/or GRAVIN activity during therapy.

Inhibitors of PARP-3 activity include, but are not limited to, phthalazin-1(2H)-ones, isoindolinones, nicotinamide, 3-aminobenzamide, benzamide, 4-amino-1,8-napthalimide, 6(5H)-Phenanthridinone, 5-aminoisoquinolinone hydrochloride, 4-hydroxyquinazoline, 4-quinazolinol, 1,5-isoquinolinediol, 5-hydroxy-1(2H)-isoquinolinone, and 3,4-dihydro-5-[4-(1-piperidinyl)butoxy]-1(2H)-isoquinolinone. Inhibitors of BCL-2A1 activity include, but are not limited to, reticulon (RTN) family proteins, sodium butyrate, antimycin A, and small molecules such as ethyl 2-amino-6-bromo-4-[1-cyano-2-ethoxy-2-oxoethyl]-4H4chromene-3-carboxylate (HA14-1). Therapeutic agents may be antiangiogenic agents or chemotherapeutic agents, for example.

Cancer cells in a subject to be treated can be any cancer that exhibits elevated ETRB activity, BCL-2A1 activity, and/or PARP-3 activity. In one embodiment, the cancer is a malignant tumor. In another embodiment, the cancer is a metastases. Cancers include, but are not limited to, the following organs or systems: cardiac, lung, gastrointestinal, genitourinary tract, liver, bone, nervous system, gynecological, hematologic, skin, and adrenal glands. Thus, the methods herein can be used for treating gliomas (Schwannoma, glioblastoma, astrocytoma), neuroblastoma, pheochromocytoma, paraganlioma, meningioma, adrenalcortical carcinoma, kidney cancer, vascular cancer of various types, osteoblastic osteocarcinoma, prostate cancer, ovarian cancer, uterine leiomyomas, salivary gland cancer, choroid plexus carcinoma, mammary cancer, pancreatic cancer, colon cancer, and megakaryoblastic leukemia. Skin cancer includes malignant melanoma, basal cell carcinoma, squamous cell carcinoma, Karposi's sarcoma, moles dysplastic nevi, lipoma, angioma, dermatofibroma, keloids, and psoriasis. In one embodiment, the cancer is metastatic melanoma. The agents of the invention can be administered in any way typical of an agent used to treat the particular type of cancer. For example, the agent(s) can be administered systemically, orally or parenterally, including, for example, by injection or as a suppository, or by any combination of such methods.

Thus, in one embodiment, the invention provides a method of ameliorating a tumor in a subject. Such a method can be performed by administering to the subject a therapeutically effective amount of a selective inhibitor of BCL-2A1 activity and/or PARP-3 activity, such that the inhibitor contacts cells of the tumor in the subject. In another embodiment, the method of ameliorating a tumor in a subject involves administering to the subject a therapeutically effective amount of a nucleic acid molecule, such that the nucleic acid molecule silences endothelin receptor B activity in cells of the tumor in the subject by RNAi. In yet another embodiment, the method of ameliorating a tumor in a subject involves administering to the subject a therapeutically effective amount of a selective inhibitor of endothelin receptor B activity in combination with a therapeutic agent, such that the inhibitor and the therapeutic agent contact the cells of the tumor in the subject.

The present invention further relates to a method of identifying cancer cells of a subject amenable to the treatments as described above. As such, the method provides a means to determine whether a subject having cancer is likely to be responsive to treatment with the inhibitors of the invention. The method can be performed, for example, by detecting elevated ETRB activity in a sample of cells of the subject as compared to corresponding normal cells, wherein detection of an elevated level indicates that the subject can benefit from treatment with an inhibitor. Likewise, the method can be performed by detecting elevated levels of HIF-1α or VEGF, and/or detecting reduced levels of GRAVIN, as compared to corresponding normal cells.

The sample of cells can be any sample, including, for example, a tumor sample obtained by biopsy of a subject having the tumor, a tumor sample obtained by surgery (e.g., a surgical procedure to remove and/or debulk the tumor), or a sample of the subject's bodily fluid.

In one embodiment, the method of identifying cancer cells of a subject amenable to treatment includes detecting elevated endothelin receptor B activity in a sample of cells from the subject as compared to endothelin receptor B activity in corresponding normal cells, thereby identifying cancer cells of a subject amenable to treatment with a nucleic acid molecule that results in silencing endothelin receptor B activity through RNAi. The method may further include contacting the cells with a nucleic acid molecule that results in silencing endothelin receptor B activity through RNAi, and detecting a decrease in endothelin receptor B activity following the contact, thereby confirming that the cancer cells are amenable to treatment with a nucleic acid molecule that results in silencing endothelin receptor B activity through RNAi.

In another embodiment, the method of identifying cancer cells of a subject amenable to treatment includes detecting elevated endothelin receptor B activity in a sample of cells from the subject as compared to endothelin receptor B activity in corresponding normal cells, thereby identifying cancer cells of a subject amenable to treatment with a selective inhibitor of BCL-2A1 activity or PARP-3 activity. The method may further include contacting the cells with a selective inhibitor of BCL-2A1 activity or PARP-3 activity, and detecting a decrease in endothelin B receptor activity following the contact, thereby confirming that the cancer cells are amenable to treatment with a selective inhibitor of BCL-2A1 activity or PARP-3 activity.

In another embodiment, the method of identifying cancer cells of a subject amenable to treatment includes contacting the cells with a selective inhibitor of an endothelin receptor B activity and detecting an increase in angiogenesis in a sample of cells from the subject as compared to the level of angiogenesis in corresponding normal cells, thereby identifying cancer cells of a subject amenable to treatment with a selective inhibitor of endothelin receptor B activity in combination with a therapeutic agent. The method may further include detecting elevated levels of HIF-1α activity or VEGF activity in the sample of cells, as compared to HIF-1α activity or VEGF activity in corresponding normal cells or untreated cancer cells. Likewise, the method may further include detecting decreased levels of GRAVIN activity in the sample of cells, as compared to GRAVIN activity in corresponding normal cells or untreated cancer cells. Additionally, the methods of identifying cancer cells of a subject amenable to treatment may further include contacting the cells with a therapeutic agent, and detecting apoptosis following the contact, thereby confirming that the cancer cells are amenable to treatment with a selective inhibitor of endothelin receptor B activity in combination with a therapeutic agent.

The present invention further relates to a method of identifying an agent useful for treating cancer in combination with a selective inhibitor of an endothelin receptor B activity. In one embodiment, the method provides a means for practicing personalized medicine, wherein treatment is tailored to the particular subject based on the characteristics of the cancer cells in the subject. The present method can be practiced, for example, by contacting a sample of cells of cancer cells with at least one test agent in combination with a selective inhibitor of endothelin receptor B activity, wherein detection of apoptosis following the contact identifies the agent as useful for treating cancer.

The present method can be practiced using agents that are known to be effective in treating cancer in order to identify one or more agents that are particularly useful for treating the cancer being examined, or using agents that are being examined for effectiveness. As such, in one aspect, the candidate agent examined according to the present method can be any type of compound, including, for example, a peptide, a polynucleotide, a peptidomimetic, or a small organic molecule, and can be one of a plurality of similar but different agents (e.g., a combinatorial library of test agents, which can be a randomized or biased library or can be a variegated library based on known effective agent).

Generally, though not necessarily, the method is performed by contacting the sample of cells ex vivo, for example, in a culture medium or on a solid support. As such, the methods are conveniently adaptable to a high throughput format, wherein a plurality (i.e., 2 or more) of samples of cells, which can be the same or different, are examined in parallel. Thus in one embodiment, candidate agents can be tested on several samples of cells from a single subject, allowing, for example, for the identification of a particularly effective concentration of an agent to be administered to the subject, or for the identification of a particularly effective agent to be administered to the subject. In another embodiment, a high throughput format allows for the examination of two, three, four, etc., different test agents, alone or in combination, on the cancer cells of a subject such that the best (most effective) agent or combination of agents can be used for a therapeutic procedure. Accordingly, in various embodiments, the high throughput method is practiced by contacting different samples of cells of different subjects with same amounts of a candidate agent; or contacting different samples of cells of a single subject with different amounts of a candidate agent; or contacting different samples of cells of two or more different subjects with same or different amounts of different candidate agents. Further, a high throughput format allows, for example, control samples (positive controls and or negative controls) to be run in parallel with test samples, including, for example, samples of cells known to be effectively treated with an agent being tested. Variations of the exemplified methods also are contemplated.

### BRIEF DESCRIPTION OF THE FIGURES

FIGS. 1A-1I show pictorial representations and graphical diagrams indicating that BQ788 is effective against metastatic melanoma. Cells were cultured on a matrigel matrix that was histologically examined after two weeks (A-F). While the primary melanoma cells remained on top of the gel only (A and B), a fraction of the Cut-met cells invaded the gel (C and D) and the LN-met cells invaded the matrix extensively (E and F). Cell lysates of the three lines were subjected to western blot analysis using an anti-ETRB Ab (G) and an anti-tubulin Ab (H). Untreated, control (vehicle treated), and BQ788 treated cells were subjected to MTS assay to quantify any reduction in cell number. The mean values were calculated and plotted (I) as the percentage of the vehicle treated value+/− SEM. BQ788 values for the three cell lines were significantly different than control (p<0.05).

FIGS. 2A-F are graphical diagrams indicating that BQ788 induces apoptosis. Equal amounts of RNA from cells that were treated with BQ788 for 3 days were subjected to real-time RT-PCR analysis for the detection of BCL-2A1 (A) and ADP ribosyltransferase 3 (B) transcripts. Total amounts are represented in A while relative (to control) values are shown in B. Differences in expression levels are shown as x fold decrease and are statistically significant (p<0.05) unless a column in marked as non significant (N.S.). Lysates of BQ788 treated cells and controls were subjected to an apoptosis ELISA detection test (C). SK-MEL-28 cells were cultured with pan caspase inhibitor (D) or with caspase 6 inhibitor (E) with or without BQ788 for 2.5 days. Only BQ788 values are significantly different than controls (p<0.05). SK-MEL-28 cells were cultured for 3 days in the presence of BQ788 and assayed for caspase 6 activity (F). The difference between the columns is statistically significant (p<0.05).

FIGS. 3A and 3B show pictorial representations and graphical diagrams indicating that BQ788 decreases ETRB RNA (A) and protein (B) expression levels. Real-time RT-PCR detection of ETRB transcripts (see legends FIG. 2) (A). ETRB protein expression compared to tubulin in SK-MEL-28 cells after 3, 5 and 7 days with (+) or without (−) BQ788 (B).

FIGS. 4A-C show pictorial representations and graphical diagrams indicating that siRNA-mediated reduction in ETRB (EDNRB) protein levels (A) result in reduced melanoma viability (B). SK-MEL-28 cells were transiently transfected either with empty plasmid (pSuper), a control construct containing siRNA sequences for the Calpain gene, or siRNA sequences targeting ETRB (EDNRB) for 24 or 48 h and subjected to western blot analysis for the detection of ETRB (top) and tubulin (bottom) protein content (A). The plasmid that was used to transfect the cells in the different lanes is indicated between the ETRB and tubulin blots. Viability of SK-MEL-28 (B) or LN-met and Cut-met (C) transfected cells was measured using an MTS test after 48 h. Significant differences (p<0.05) from control (pSuper) are indicated with a value showing the degree of viability reduction as opposed to not significant (N.S.) difference.

FIGS. 5A-E show pictorial representations and graphical diagrams indicating that BQ788 increases angiogenesis. Equal amounts of RNA from cells that were treated with BQ788 for 3 days were subjected to real-time RT-PCR analysis for the detection VEGF (A), HIF-1α (B) and Gravin (C) transcripts. Differences in expression levels are shown as x fold change and are statistically significant (p<0.05) unless a column in marked as non significant (N.S.). Sections of two representative tumors derived from a human melanoma cell line grown in nude mice and injected either with vehicle (D) or BQ788 (E) were stained with anti CD-31 to highlight blood vessels (brown staining, Lahav et al, 1999).

FIG. 6 is a graphical diagram showing the effect of Glioma cell lines treated with BQ788.

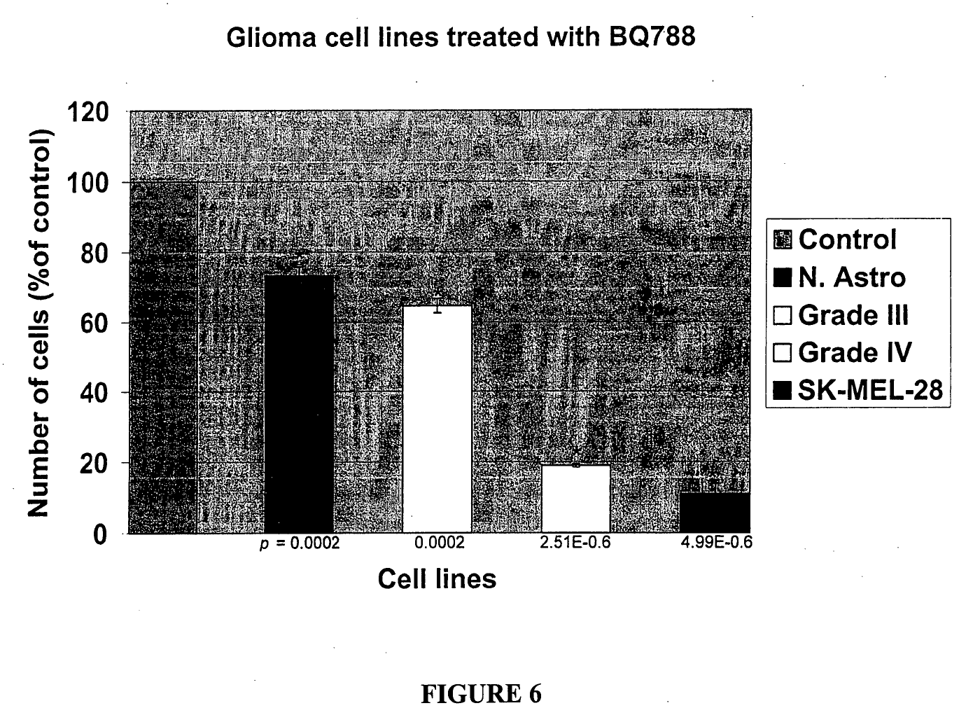

### DETAILED DESCRIPTION OF THE INVENTION

The present invention relates to the discovery that inhibition of the abnormal proliferation of cells is useful in treating a number of disorders such as cancer, autoimmune disease, arthritis, inflammatory bowel disease, proliferation induced after medical procedures, and many other instances. In particular, the invention is based, in part, on the determination that endothelin receptor B (ETRB) activity and/or BCL-2A1 activity and/or PARP-3 activity is elevated in cancer cells as compared to corresponding normal cells, and that agents that decrease ETRB activity and/or BCL-2A1 and/or PARP-3 activity inhibit proliferation and/or induce cell death of cancer cells.

The present invention is not limited to the particular methodology, protocols, cell lines, vectors, reagents, etc., described herein, as these may vary. It is also to be understood that the terminology used herein is used for the purpose of describing particular embodiments only, and is not intended to limit the scope of the present invention. As used herein and in the appended claims, the singular forms “a,” “an,” and “the” include plural reference unless the context clearly dictates otherwise.

In one aspect of the invention, the subject methods can be used as part of a treatment regimen for cancer. In some cases, the treatment of cancer may include the treatment of solid tumors or the treatment of metastasis. Metastasis is a form of cancer wherein the transformed or malignant cells are traveling and spreading the cancer from one site to another. Such cancers include cancers of the skin, breast, brain, cervical carcinomas, testicular carcinomas, etc. More particularly, cancers may include, but are not limited to the following organs or systems: cardiac, lung, gastrointestinal, genitourinary tract, liver, bone, nervous system, gynecological, hematologic, skin, and adrenal glands. More particularly, the methods herein can be used for treating gliomas (Schwannoma, glioblastoma, astrocytoma), neuroblastoma, pheochromocytoma, paraganlioma, meningioma, adrenalcortical carcinoma, kidney cancer, vascular cancer of various types, osteoblastic osteocarcinoma, prostate cancer, ovarian cancer, uterine leiomyomas, salivary gland cancer, choroid plexus carcinoma, mammary cancer, pancreatic cancer, colon cancer, and megakaryoblastic leukemia. Skin cancer includes malignant melanoma, basal cell carcinoma, squamous cell carcinoma, Karposi's sarcoma, moles dysplastic nevi, lipoma, angioma, dermatofibroma, keloids, and psoriasis. In one embodiment, the cancer is metastatic melanoma.

The term “cancer” as used herein, includes any malignant tumor including, but not limited to, carcinoma, sarcoma. Cancer arises from the uncontrolled and/or abnormal division of cells that then invade and destroy the surrounding tissues. As used herein, “proliferating” and “proliferation” refer to cells undergoing mitosis. As used herein, “metastasis” refers to the distant spread of a malignant tumor from its sight of origin. Cancer cells may metastasize through the bloodstream, through the lymphatic system, across body cavities, or any combination thereof.

The term “cancerous cell” as provided herein, includes a cell afflicted by any one of the cancerous conditions provided herein. Thus, the methods of the present invention include treatment of benign overgrowth of melanocytes, glia, prostate hyperplasia, and polycystic kidney disease. The term “carcinoma” refers to a malignant new growth made up of epithelial cells tending to infiltrate surrounding tissues, and to give rise to metastases.

The term “subject” as used herein refers to any individual or patient to which the subject methods are performed. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject. However, the method can also be practiced in other species, such as avian species (e.g., chickens).

The term “therapeutically effective amount” or “effective amount” means the amount of a compound or pharmaceutical composition that will elicit the biological or medical response of a tissue, system, animal or human that is being sought by the researcher, veterinarian, medical doctor or other clinician.

The term “pharmaceutically acceptable”, when used in reference to a carrier, is meant that the carrier, diluent or excipient must be compatible with the other ingredients of the formulation and not deleterious to the recipient thereof.

The terms “administration” or “administering” is defined to include an act of providing a compound or pharmaceutical composition of the invention to a subject in need of treatment. The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrastemal injection and infusion. The phrases “systemic administration,” “administered systemically,” “peripheral administration” and “administered peripherally” as used herein mean the administration of a compound, drug or other material other than directly into the central nervous system, such that it enters the subject's system and, thus, is subject to metabolism and other like processes, for example, subcutaneous administration.

The term “agonist” refers to an agent or analog that binds productively to a receptor and mimics its biological activity. The term “antagonist” refers to an agent that binds to receptors but does not provoke the normal biological response. For example, an antagonist may be any molecule which, when bound to an endothelin receptor (ERT), decreases the activity of or reduces the expression levels of the ERT. In melanoma (and glioma) in particular, reduced expression of ETRB appears to occur simultaneously with reduced expression of BCL-2A1, PARP-3 and GRAVIN, and increased expression of HIF-1α and VEGF. Thus an agonist or antagonist of the invention includes any agent that results in reduced survival of melanoma (and glioma) cells by causing the combination of reduced ETRB, BCL-2A1, PARP-3 and GRAVIN, and increased HIF-1α and VEGF. Agonists or antagonists may include proteins, nucleic acids, carbohydrates, antibodies, or any other molecules which decrease the normal biological response.

The term “antibody” as used in this invention is meant to include intact molecules of polyclonal or monoclonal antibodies, as well as fragments thereof, such as Fab and F(ab′)₂, Fv and SCA fragments which are capable of binding an epitopic determinant.

The term “antisense,” as used herein, refers to any composition containing a nucleic acid sequence which is complementary to a specific nucleic acid sequence. The term “antisense strand” is used in reference to a nucleic acid strand that is complementary to the “sense” strand. Antisense molecules may be produced by any method including synthesis or transcription. Once introduced into a cell, the complementary nucleotides combine with natural sequences produced by the cell to form duplexes and to block either transcription or translation.

As used herein, “proliferating” and “proliferation” refer to cells undergoing mitosis. The term “transformed cells” refers to cells which have spontaneously converted to a state of unrestrained growth, i.e., they have acquired the ability to grow through an indefinite number of divisions in culture. Transformed cells may be characterized by such terms as neoplastic, anaplastic and/or hyperplastic, with respect to their loss of growth control.

As used herein “corresponding normal cells” means cells that are from the same organ and of the same type as cancer cells being examined. In one aspect, the corresponding normal cells comprise a sample of cells obtained from a healthy individual. Such corresponding normal cells can, but need not be, from an individual that is age-matched and/or of the same sex as the individual providing the cancer cells being examined. In another aspect, the corresponding normal cells comprise a sample of cells obtained from an otherwise healthy portion of tissue of a subject having cancer.

As used herein, the terms “sample” and “biological sample” refer to any sample suitable for the methods provided by the present invention. In one embodiment, the biological sample of the present invention is a tissue sample, e.g., a biopsy specimen such as samples from needle biopsy. In other embodiments, the biological sample of the present invention is a sample of bodily fluid, e.g., serum, plasma, urine, and ejaculate.

As used herein, the terms “reduce” and “inhibit” are used together because it is recognized that, in some cases, a decrease, for example, in ETRB activity can be reduced below the level of detection of a particular assay. As such, it may not always be clear whether the activity is “reduced” below a level of detection of an assay, or is completely “inhibited”. Nevertheless, it will be clearly determinable, following a treatment according to the present methods, that the level of ETRB activity (and/or cell proliferation or metastasis) is at least reduced from the level before treatment.

In one aspect, the method for treating cancer provided herein includes administering to an individual or a cell, an inhibitor of endothelin receptor (ETR) activity in combination with a therapeutic agent. ETR activity includes activities which are induced by agonists to an endothelin receptor. Endothelin receptors include the endothelin receptor A (ERTA or ENDRA) and the endothelin receptor B (ERTB or ENDRB). ERTB are localized at least to the endothelium and nonvascular tissues such as the liver, kidney and brain. The receptors are also located in certain vascular smooth muscle tissues. Thus, in one embodiment, the ERTB is expressed, but at a comparatively low level compared to expansion levels in other malignant cells. Agonists of an ETR include ET1, ET2, ET3, and S6c. An ETR agonist as defined herein includes agents with the ability to enhance proliferation and/or delay differentiation.

In another embodiment, an ETRB inhibitor causes increased expression of HIF-1α and VEGF, and reduced expression of GRAVIN, all of which result in increased angiogenesis in treated tumors. Thus, an ETRB inhibitor used in combination with therapeutic agents would take advantage of increased permeability to the tumor cells. ETRB inhibitor activity includes one or more of the following characteristics: inhibits cancerous growth, regresses cancer growth, induces apoptosis in a cancerous cell, induces cell death in a cancerous cell, induces differentiation in a cancerous cell, induces pigmentation in a cancerous cell, antagonizes ET3, ET2 and/or ET1, binds to an ETR selectively, and antagonizes S6c. Any combination of these characteristics including all or one or more, with one or more exclusions, is provided herein.

Thus, an inhibitor of ETR activity as defined herein inhibits cancerous growth, or reduces proliferation, by at least 30%, more preferably 40%, more preferably 50%, more preferably 70%, more preferably 90%, and most preferably by at least 95%. In another embodiment, the ETR inhibitor causes tumor regression by at least 30%, more preferably 40%, more preferably 50%, more preferably 70%, more preferably 90%, and most preferably by at least 95%. The determination of inhibition or regression can be made by comparing the effect with treatment as described herein, compared to a control sample wherein treatment, for example, with an inhibitor of the endothelin receptor, is not provided. In some cases, the control sample may have a tumor which grows to twice, three times, or four times the volume of the tissue being treated in accordance with the methods as described herein.

Exemplary ETRB activity inhibitors, or ETRB inhibitors, include but are not limited to, BQ788 (N-cis-2,6-dimethylpiperidinocarbonyl-L-γ-methylleucyl-D-1-methoxycarbonyltrptophanyl-D-norleucine has been previously described, e.g., Ishikawa, et al., PNAS, 91:4892-4896 (1994)), IRL1083, RES7011, RES7013, PD142983, IRL2500, R0468443 and A192621. Derivatives of BQ788 as used herein are functionally equivalent to BQ788 in that they have ETR inhibitor activity. Exemplary ETRA activity inhibitors, or ETRA inhibitors, include but are not limited to, LU135252, BQ485, BQ123, FR139317, BE18257B, JKC301, JKC302, BQ610, PD156707, A127722, R061-1790, TBC11251, FR139317, S0139, A127722, SB234551, A192621, ABT627, A216546, PD155080, BMS182874, 97139, LU127043, and IRL1620.

In some instances, the ETR inhibitor binds to more than one ETR. Thus the ETR inhibitor may be LU302872, TAK044, PD142893, PD145065, BE18257A/W7338A, Bosentan (RO47-0203), SB217242, R0468443, SB209670, Tnieno [2,3-d]pyrimidine-3-aceticacids, R0610612, R0462005, PD156252, A182086, L744453, and L754142.

In one embodiment, the ETR inhibitor is an antisense molecule to the nucleic acid encoding an ETR or an ETR native ligand such as ET1, ET2, or ET3. Antisense molecules include oligonucleotides comprising a singe-stranded nucleic acid sequence (either RNA or DNA) capable of binding to target receptor or ligand mRNA (sense) or DNA (antisense) sequences. Antisense or sense oligonucleotides, according to the present invention, comprise a fragment of the coding region of the receptor or ligand. Such a fragment generally comprises at least about 14 nucleotides, preferably from about 14 to 30 nucleotides. The ability to derive an antisense or a sense oligonucleotide, based upon a cDNA sequence encoding a given protein (previously described in the art) is described in, for example, Stein, et al., Cancer Res., 48:2659, (1988) and van der Krol, et al., Bio Techniques, 6:958 (1988). Antisense or sense oligonucleotides further comprise oligonucleotides having modified sugar-phosphodiester backbones (or other sugar linkages, such as those described in WO 91/06629) and wherein such sugar linkages are resistant to endogenous nucleases. Such oligonucleotides with resistant sugar linkages are stable in vivo (i.e., capable of resisting enzymatic degradation) but retain sequence specificity to be able to bind to target nucleotide sequences.

Other examples of sense or antisense oligonucleotides include those oligonucleotides which are covalently linked to organic moieties, such as those described in WO 90/10048, and other moieties that increases affinity of the oligonucleotide for a target nucleic acid sequence, such as poly-(L-lysine). Further still, intercalating agents, such as ellipticine, and alkylating agents or metal complexes may be attached to sense or antisense oligonucleotides to modify binding specificities of the antisense or sense oligonucleotide for the target nucleotide sequence.

Antisense or sense oligonucleotides may be introduced into a cell by any gene transfer method. For example, delivery of antisense molecules and the like can be achieved using a recombinant expression vector such as a chimeric virus or a colloidal dispersion system. Various viral vectors which can be utilized for gene therapy as taught herein include adenovirus, herpes virus, vaccinia or preferably an RNA virus such as a retrovirus. A number of the known retroviruses can transfer or incorporate a gene for a selectable marker so that transduced cells can be identified and generated. By inserting a polynucleotide sequence of interest into the viral vector, along with another gene which encodes the ligand for a receptor on a specific target cell, for example, the vector is target specific. Retroviral vectors can be made target specific by inserting, for example, a polynucleotide encoding a sugar, a glycolipid or a protein. Preferred targeting is accomplished by using an antibody to target the retroviral vector. Those of skill in the art will know of, or can readily ascertain without undue experimentation, specific polynucleotide sequences which can be inserted into the retroviral genome to allow target specific delivery of the retroviral vector containing the antisense polynucleotide

In another embodiment, the ETR inhibitor is an inhibitor of the endothelin converting enzyme (ECE) which processes endothelin precursors. ECE inhibitors include but are not limited to CGS26303 and phosphoramidon. ECE antisense molecules can also be used. In yet another embodiment, compositions that inhibit growth factor receptors in the same family as ETRB are also provided herein for methods of treatment.

In another aspect, the present invention provides a method of ameliorating or treating a tumor in a subject with the subject inhibitors. As used herein, the term “ameliorating” or “treating” means that the clinical signs and/or the symptoms associated with the cancer or melanoma are lessened as a result of the actions performed. The signs or symptoms to be monitored will be characteristic of a particular cancer or melanoma and will be well known to the skilled clinician, as will the methods for monitoring the signs and conditions. For example, the skilled clinician will know that the size or rate of growth of a tumor can monitored using a diagnostic imaging method typically used for the particular tumor (e.g., using ultrasound or magnetic resonance image (MRI) to monitor a tumor).

In one embodiment, the method for treating cancer includes administering to the subject a therapeutically effective amount of a nucleic acid molecule, such as double-stranded RNA (dsRNA), in order to induce RNA interference (RNAi) and silence ETRB activity. RNAi is a phenomenon in which the introduction of dsRNA into a diverse range of organisms and cell types causes degradation of the complementary mRNA. In the cell, long dsRNAs are cleaved into short (e.g., 21-25 nucleotide) small interfering RNAs (siRNAs), by a ribonuclease. The siRNAs subsequently assemble with protein components into an RNA-induced silencing complex (RISC), unwinding in the process. The activated RISC then binds to complementary transcripts by base pairing interactions between the siRNA antisense strand and the mRNA. The bound mRNA is then cleaved and sequence specific degradation of mRNA results in gene silencing. As used herein, “silencing” refers to a mechanism by which cells shut down large sections of chromosomal DNA resulting in suppressing the expression of a particular gene. The RNAi machinery appears to have evolved to protect the genome from endogenous transposable elements and from viral infections. Thus, RNAi can be induced by introducing nucleic acid molecules complementary to the target mRNA to be degraded, as described in the examples below.

Classes of therapeutic agents suitable for use in methods of the present invention include, but are not limited to: 1) alkaloids, including, microtubule inhibitors (e.g., Vincristine, Vinblastine, and Vindesine, etc.), microtubule stabilizers (e.g., Paclitaxel [Taxol], and Docetaxel, Taxotere, etc.), and chromatin function inhibitors, including, topoisomerase inhibitors, such as, epipodophyllotoxins (e.g., Etoposide [VP-16], and Teniposide [VM-26], etc.), and agents that target topoisomerase I (e.g., Camptothecin and Isirinotecan [CPT-11], etc.); 2) covalent DNA-binding agents [alkylating agents], including, nitrogen mustards (e.g., Mechlorethamine, Chlorambucil, Cyclophosphamide, Ifosphamide, and Busulfan [Myleran], etc.), nitrosoureas (e.g., Carmustine, Lomustine, and Semustine, etc.), and other alkylating agents (e.g., Dacarbazine, Hydroxymethylmelamine, Thiotepa, and Mitocycin, etc.); 3) noncovalent DNA-binding agents [antitumor antibiotics], including, nucleic acid inhibitors (e.g., Dactinomycin [Actinomycin D], etc.), anthracyclines (e.g., Daunorubicin [Daunomycin, and Cerubidine], Doxorubicin [Adriamycin], and Idarubicin [Idamycin], etc.), anthracenediones (e.g., anthracycline analogues, such as, [Mitoxantrone], etc.), bleomycins (Blenoxane), etc., and plicamycin (Mithramycin), etc.; 4) antimetabolites, including, antifolates (e.g., Methotrexate, Folex, and Mexate, etc.), purine antimetabolites (e.g., 6-Mercaptopurine [6-MP, Purinethol], 6-Thioguanine [6-TG], Azathioprine, Acyclovir, Ganciclovir, Chlorodeoxyadenosine, 2-Chlorodeoxyadenosine [CdA], and 2′-Deoxycoformycin [Pentostatin], etc.), pyrimidine antagonists (e.g., fluoropyrimidines [e.g., 5-fluorouracil (Adrucil), 5-fluorodeoxyuridine (FdUrd) (Floxuridine)] etc.), and cytosine arabinosides (e.g., Cytosar [ara-C] and Fludarabine, etc.); 5) enzymes, including, L-asparaginase, and hydroxyurea, etc.; 6) hormones, including, glucocorticoids, such as, antiestrogens (e.g., Tamoxifen, etc.), nonsteroidal antiandrogens (e.g., Flutamide, etc.), and aromatase inhibitors (e.g., anastrozole [Arimidex], etc.); 7) platinum compounds (e.g., Cisplatin and Carboplatin, etc.); 8) monoclonal antibodies conjugated with anticancer drugs, toxins, and/or radionuclides, etc.; 9) biological response modifiers (e.g., interferons [e.g., IFN-α, etc.] and interleukins [e.g., IL-2, etc.], etc.); 10) adoptive immunotherapy; 11) hematopoietic growth factors; 12) agents that induce tumor cell differentiation (e.g., all-trans-retinoic acid, etc.); 13) gene therapy techniques; 14) antisense therapy techniques; 15) tumor vaccines; 16) therapies directed against tumor metastases (e.g., Batimistat, etc.); 17) antiangiogenic agents; 18) chemotherapeutic agents and 19) known treatmens to reduce blood pressure.

In yet another embodiment, the method for treating cancer includes administering to the subject a therapeutically effective amount of a selective inhibitor of BCL-2A1 activity or PARP-3 activity. An inhibitor of BCL-2A1 activity or PARP-3 activity as defined herein inhibits cancerous growth, reduces proliferation, or induces cancer cell apoptosis, by at least 30%, more preferably 40%, more preferably 50%, more preferably 70%, more preferably 90%, and most preferably by at least 95%. In another embodiment, a BCL-2A1 activity or PARP-3 activity inhibitor causes tumor regression by at least 30%, more preferably 40%, more preferably 50%, more preferably 70%, more preferably 90%, and most preferably by at least 95%. The determination of inhibition or regression can be made by comparing the effect with treatment as described herein, compared to a control sample wherein treatment, for example, with an inhibitor of BCL-2A1 activity or PARP-3 activity, is not provided. In some cases, the control sample may have a tumor which grows to twice, three times, or four times the volume of the tissue being treated in accordance with the methods as described herein.

As used herein, “apoptosis” refers to a genetically determined process of cell self-destruction that is marked by the fragmentation of nuclear DNA, and is activated either by the presence of a stimulus or by the removal of a stimulus or suppressing agent. A characteristic feature of apoptosis is activation of a cascade of cytoplasmic proteases that results in the cleavage of selected target proteins. Standard kits for identifying cells undergoing apoptosis, for example, the TUNEL method, are known in the art. Additionally, apoptosis can be identified by a significant increase in hypodiploid cells, chromatin condensation and/or DNA fragmentation.

Exemplary PARP-3 inhibitors, include but are not limited to, phthalazin-1(2H)-ones, isoindolinones, nicotinamide, 3-aminobenzamide, benzamide, 4-amino-1,8-napthalimide, 6(5H)-Phenanthridinone, 5-aminoisoquinolinone hydrochloride, 4-hydroxyquinazoline, 4-quinazolinol, 1,5-isoquinolinediol, 5-hydroxy-1(2H)-isoquinolinone, and 3,4-dihydro-5-[4-(1-piperidinyl)butoxy]-1(2H)-isoquinolinone.

Exemplary BCL-2A1 inhibitors, include but are not limited to, reticulon (RTN) family proteins, sodium butyrate, antimycin A, and small molecules such as ethyl 2-amino-6-bromo-4-[1-cyano-2-ethoxy-2-oxoethyl]-4H4chromene-3-carboxylate (HA14-1).

The pharmaceutical compositions of the present invention may further comprise other therapeutically active compounds as noted herein which are usually applied in the treatment of the above mentioned pathological conditions. Examples of other therapeutic agents include the following: cyclosporins (e.g., cyclosporin A), CTLA4-Ig, antibodies such as ICAM-3, anti-IL-2 receptor (Anti-Tac), anti-CD45RB, anti-CD2, anti-CD3 (OKT-3), anti-CD4, anti-CD80, anti-CD86, agents blocking the interaction between CD40 and gp39, such as antibodies specific for CD40 and/or gp39 (i.e., CD154), fusion proteins constructed from CD40 and gp39 (CD40Ig and CD8gp39), inhibitors, such as nuclear translocation inhibitors, of NF-κB function, such as deoxyspergualin (DSG), cholesterol biosynthesis inhibitors such as HMG CoA reductase inhibitors (lovastatin and simvastatin), non-steroidal antiinflammatory drugs (NSAIDs) such as ibuprofen and cyclooxygenase inhibitors such as rofecoxib, steroids such as prednisone or dexamethasone, gold compounds, antiproliferative agents such as methotrexate, FK506 (tacrolimus, Prograf), mycophenolate mofetil, cytotoxic drugs such as azathioprine and cyclophosphamide, VEGF inhibitors, TNF-a inhibitors such as tenidap, anti-TNF antibodies or soluble TNF receptor, and rapamycin (sirolimus or Rapamune) or derivatives thereof.

The invention compounds may further be administered in combination with an antiinflammatory, antihistamine, chemotherapeutic agent, antiangiogenic agent, immunomodulator, therapeutic antibody or a protein kinase inhibitor, e.g., a tyrosine kinase inhibitor, to a subject in need of such treatment. While not wanting to be limiting, chemotherapeutic agents include antimetabolites, such as methotrexate, DNA cross-linking agents, such as cisplatin/carboplatin; alkylating agents, such as canbusil; topoisomerase I inhibitors such as dactinomicin; microtubule inhibitors such as taxol (paclitaxol), and the like. Other chemotherapeutic agents include, for example, a vinca alkaloid, mitomycin-type antibiotic, bleomycin-type antibiotic, antifolate, colchicine, demecoline, etoposide, taxane, anthracycline antibiotic, doxorubicin, daunorubicin, carminomycin, epirubicin, idarubicin, mithoxanthrone, 4-demethoxy-daunomycin, 11-deoxydaunorubicin, 13-deoxydaunorubicin, adriamycin-14-benzoate, adriamycin-14-octanoate, adriamycin-14-naphthaleneacetate, amsacrine, carmustine, cyclophosphamide, cytarabine, etoposide, lovastatin, melphalan, topetecan, oxalaplatin, chlorambucil, methtrexate, lomustine, thioguanine, asparaginase, vinblastine, vindesine, tamoxifen, or mechlorethamine. While not wanting to be limiting, antiangiogenic agents include, for example, thalidomide, rofecoxib, celecoxib, bevacizumab, angiostatin, and endostatin. While not wanting to be limiting, therapeutic antibodies include antibodies directed against the HER2 protein, such as trastuzumab; antibodies directed against growth factors or growth factor receptors, such as bevacizumab, which targets vascular endothelial growth factor, and OSI-774, which targets epidermal growth factor; antibodies targeting integrin receptors, such as Vitaxin (also known as MEDI-522), and the like.

Other agents that may be administered in combination with invention compounds include protein therapeutic agents such as cytokines, immunomodulatory agents and antibodies. As used herein the term “cytokine” encompasses chemokines, interleukins, lymphokines, monokines, colony stimulating factors, and receptor associated proteins, and functional fragments thereof. As used herein, the term “functional fragment” refers to a polypeptide or peptide which possesses biological function or activity that is identified through a defined functional assay.

The cytokines include endothelial monocyte activating polypeptide II (EMAP-II), granulocyte-macrophage-CSF (GM-CSF), granulocyte-CSF (G-CSF), macrophage-CSF (M-CSF), IL-1, IL-2, IL-3, IL-4, IL-5, IL-6, IL-12, and IL-13, interferons, and the like and which is associated with a particular biologic, morphologic, or phenotypic alteration in a cell or cell mechanism.

When other therapeutic agents are employed in combination with the compounds of the present invention they may be used for example in amounts as noted in the Physician Desk Reference (PDR) or as otherwise determined by one having ordinary skill in the art.

The invention also provides a method of determining whether cancer cells are amenable to treatments of the invention. The method can be performed, for example, by measuring the level of ETRB activity in a sample of cells to be treated, and determining ETRB activity is elevated as compared to the level of ETRB activity in corresponding normal cells, which can be a sample of normal (i.e., not tumor) cells. Detection of elevated levels of ETRB activity in the cancer cells as compared to the corresponding normal cells indicates that the cells can benefit from treatment. A sample of cells used in the present method can be obtained from tissue samples or bodily fluid from a subject, or tissue obtained by a biopsy procedure (e.g., a needle biopsy) or a surgical procedure to remove and/or debulk the tumor.

Elevated ETRB activity can be determined by measuring elevated expression of one or more (e.g., 1, 2, 3, or more) ETRB-related polypeptide(s), including, for example, endothelin B, BCL-2A1, PARP-3, or GRAVIN, or a combination of such polypeptides. The elevated expression can be detected by measuring the level of a polynucleotide encoding the ETRB-related polypeptides (e.g., RNA) using, for example, a hybridization assay, a primer extension assay, or a polymerase chain reaction (PCR) assay (e.g., a reverse transcription-PCR assay); or by measuring the level the ETRB-related polypeptide(s) using, for example, an immunoassay or receptor binding assay. Alternatively, or in addition, elevated activity of one or more (e.g., 1, 2, 3, or more) ETRB-related polypeptide(s) can be determined. Expression of an ETRB-related polypeptide having an inactivating mutation can be identified using, for example, an antibody that specifically binds to the mutant, but not to the normal (wild type), ETRB-related polypeptide, wherein the mutation is associated with elevated ETRB activity. In addition, elevated ETRB activity can be determined by measuring decreased expression of HIF-1α activity and/or VEGF activity. For example, while ETRB inhibition reduces survival of cancer cells, melanoma cells (and glioma), in particular, have demonstrated simultaneous reduction of BCL-2A1, PARP-3, GRAVIN and increased HIF-1α and VEGF. Thus, levels of expression of one or more of BCL-2A1, PARP-3, GRAVIN, HIF-1α and VEGF may be used to determine expression levels of ETRB and vice versa.

In one embodiment, the method of identifying cancer cells amenable to treatment can further include contacting the cells with a nucleic acid molecule, such as a dsRNA, in order to induce RNAi and silence ETRB activity, and detecting decreased ETRB activity in the cells following said contact. The decreased ETRB activity can be detected, for example, by measuring decreased expression of a reporter gene regulated by ETRB, or by detecting a decrease in proliferation of the cancer cells. Such a method provides a means to confirm that the cancer cells are amenable to such treatment. Further, the method can include testing one or more different nucleic acid molecules, either alone or in combination, thus providing a means to identify one or more nucleic acid molecules useful for treating the particular cancer being examined.

In another embodiment, the method of identifying cancer cells amenable to treatment can further include contacting the cells with a selective inhibitor of BCL-2A1 activity or PARP-3 activity, and detecting a decrease in ETRB activity or GRAVIN activity, and/or an increase HIF-1α activity or VEGF activity in the cells following said contact. In this embodiment, the cancer cells are melanoma cells. The decreased activities can be detected, for example, by measuring decreased expression of a reporter gene regulated by ETRB activity or GRAVIN activity, or by detecting a decrease in proliferation of the melanoma cells. Likewise, the increased activities can be detected, for example, by measuring increased expression of a reporter gene regulated by HIF-1α activity or VEGF activity, or by detecting a decrease in proliferation of the melanoma cells. Such a method provides a means to confirm that the melanoma cells are amenable to such treatment. In another embodiment, the method can include testing one or more different inhibitors of ETRB activity, BCL-2A1 activity or PARP-3 activity, either alone or in combination, thus providing a means to identify one or more selective inhibitor of BCL-2A1 activity or PARP-3 activity useful for treating the cancer being examined.

In yet another embodiment, the method of identifying cancer cells amenable to treatment can further include contacting the cells with an inhibitor of ETRB activity in combination with a therapeutic agent. In all embodiments, detecting a decrease in ETRB activity following contacting the cells with any of the above agents is indicative of cancer cells that are amenable to treatments of the invention.

In another aspect of the invention, a method for identifying an agent useful for treating cancer in combination with a selective inhibitor of ETRB activity is provided. An agent useful in any of the methods of the invention can be any type of molecule, for example, a polynucleotide, a peptide, a peptidomimetic, peptoids such as vinylogous peptoids, a small organic molecule, or the like, and can act in any of various ways to further reduce or inhibit elevated ETRB activity, BCL-2A1 activity, PARP-3 activity, and/or GRAVIN activity when used in combination with a known inhibitor of ETRB activity. Likewise, the agent can be used to increase HIF-1α activity and/or VEGF activity when used in combination with a known inhibitor of ETRB activity. The agent can be administered in any way typical of an agent used to treat the particular type of cancer or under conditions that facilitate contact of the agent with the target tumor cells and, if appropriate, entry into the cells. Entry of a polynucleotide agent into a cell, for example, can be facilitated by incorporating the polynucleotide into a viral vector that can infect the cells. If a viral vector specific for the cell type is not available, the vector can be modified to express a receptor (or ligand) specific for a ligand (or receptor) expressed on the target cell, or can be encapsulated within a liposome, which also can be modified to include such a ligand (or receptor). A peptide agent can be introduced into a cell by various methods, including, for example, by engineering the peptide to contain a protein transduction domain such as the human immunodeficiency virus TAT protein transduction domain, which can facilitate translocation of the peptide into the cell. Generally, an agent is formulated in a composition (e.g., a pharmaceutical composition) suitable for administration to the subject, which can be any vertebrate subject, including a mammalian subject (e.g., a human subject). Such formulated agents are useful as medicaments for treating a subject suffering from cancer that is characterized, in part, by elevated ETRB activity.

Candidate agents encompass numerous chemical classes, though typically they are organic molecules, preferably small organic compounds (i.e., small molecules) having a molecular weight of more than 100 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

In other embodiments, the candidate agents are peptides of from about 5 to about 30 amino acids, with from about 5 to about 20 amino acids being preferred, and from about 7 to about 15 being particularly preferred. The peptides may be digests of naturally occurring proteins as is outlined above, random peptides, or “biased” random peptides. By “randomized” or grammatical equivalents herein is meant that each nucleic acid and peptide consists of essentially random nucleotides and amino acids, respectively. Since generally these random peptides are chemically synthesized, they may incorporate any nucleotide or amino acid at any position. The synthetic process can be designed to generate randomized proteins or nucleic acids, to allow the formation of all or most of the possible combinations over the length of the sequence, thus forming a library of randomized candidate bioactive proteinaceous agents.

By “protein” herein is meant at least two covalently attached amino acids, which includes proteins, polypeptides, oligopeptides and peptides. A protein may be made up of naturally occurring amino acids and peptide bonds, or synthetic peptidomimetic structures. Thus “amino acid”, or “peptide residue”, as used herein means both naturally occurring and synthetic amino acids. For example, homo-phenylalanine, citrulline and noreleucine are considered amino acids for the purposes of the invention. “Amino acid” also includes imino acid residues such as proline and hydroxyproline. The side chains may be in either the (R) or the (S) configuration.

By “nucleic acid” or “oligonucleotide” or grammatical equivalents herein is meant at least two nucleotides covalently linked together. A nucleic acid will generally contain phosphodiester bonds, although in some cases, as outlined below, nucleic acid analogs are included that may have alternate backbones, comprising, for example, phosphoramide (Beaucage, et al., Tetrahedron, 49(10):1925 (1993) and references therein; Letsinger, J. Org. Chem., 35:3800 (1970); Sprinzl, et al., Eur. J. Biochem., 81:579 (1977); Letsinger, et al., Nucl. Acids Res., 14:3487 (1986); Sawai, et al., Chem. Lett., 805 (1984), Letsinger, et al., J. Am. Chem. Soc., 110:4470 (1988); and Pauwels, et al., Chemica Scripta, 26:141 (1986)), phosphorothioate (Mag, et al., Nucleic Acids Res., 19:1437 (1991); and U.S. Pat. No. 5,644,048), phosphorodithioate (Briu, et al., J. Am. Chem. Soc., 111:2321 (1989)), O-methylphophoroamidite linkages (see Eckstein, Oligonucleotides and Analogues: A Practical Approach, Oxford University Press), and peptide nucleic acid backbones and linkages (see Egholm, J. Am. Chem. Soc., 114:1895 (1992); Meier, et al., Chem. Int. Ed. Engl., 31:1008 (1992); Nielsen, Nature, 365:566 (1993); Carlsson, et al., Nature, 380:207 (1996), all of which are incorporated by reference)). Other analog nucleic acids include those with positive backbones (Denpcy, et al., Proc. Natl. Acad. Sci. USA, 92:6097 (1995)); non-ionic backbones (U.S. Pat. Nos. 5,386,023; 5,637,684; 5,602,240; 5,216,141; and 4,469,863; Kiedrowshi, et al., Angew. Chem. Intl. Ed. English, 30:423 (1991); Letsinger, et al., J. Am. Chem. Soc., 110:4470 (1988); Letsinger, et al., Nucleoside &Nucleotide, 13:1597 (1994); Chapters 2 and 3, ASC Symposium Series 580, “Carbohydrate Modifications in Antisense Research”, Ed. Y. S. Sanghui and P. Dan Cook; Mesmaeker, et al., Bioorganic &Medicinal Chem. Lett., 4:395 (1994); Jeffs, et al., J. Biomolecular NMR, 34:17 (1994); Tetrahedron Lett., 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins, et al., Chem. Soc. Rev., (1995) pp. 169-176). Several nucleic acid analogs are described in Rawls, C & E News, Jun. 2, 1997, page 35. All of these references are hereby expressly incorporated by reference. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, etc.

Candidate agents may be obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs.

The methods of the invention are useful for providing a means for practicing personalized medicine, wherein treatment is tailored to a subject based on the particular characteristics of the cancer cells in the subject. The method can be practiced, for example, by contacting a sample of cells from the subject with at least one test agent, wherein a decrease in ETRB activity, BCL-2A1 activity and/or PARP-3 activity in the presence of the test agent as compared to ETRB activity, BCL-2A1 activity and/or PARP-3 activity in the absence of the test agent identifies the agent as useful for treating the cancer. The sample of cells examined according to the present method can be obtained from the subject to be treated, or can be cells of an established cancer cell line of the same type as that of the subject. In one aspect, the established cancer cell line can be one of a panel of such cell lines, wherein the panel can include different cell lines of the same type of cancer and/or different cell lines of different cancers. Such a panel of cell lines can be useful, for example, to practice the present method when only a small number of cancer cells can be obtained from the subject to be treated, thus providing a surrogate sample of the subject's cancer, and also can be useful to include as control samples in practicing the present methods.

Preferred cell types for use in the invention include, but are not limited to, mammalian cells, including animal (rodents, including mice, rats, hamsters and gerbils), primates, and human cells, particularly cancer cells of all types, including breast, skin, lung, cervix, colorectal, leukemia, brain, etc.

Once disease is established and a treatment protocol is initiated, the methods of the invention may be repeated on a regular basis to evaluate whether any of the levels of ERTB activity, BCL-2A1 activity, PARP-3 activity, HIF-1α activity, VEGF activity and/or GRAVIN activity in the subject begins to approximate that which is observed in a normal subject. The results obtained from successive assays may be used to show the efficacy of treatment over a period ranging from several days to months. Accordingly, the invention is also directed to methods for monitoring a therapeutic regimen for treating a subject having cancer. A comparison of any of the levels of ERTB activity, BCL-2A1 activity, PARP-3 activity, HIF-1α activity, VEGF activity and/or GRAVIN activity prior to and during therapy indicates the efficacy of the therapy. Therefore, one skilled in the art will be able to recognize and adjust the therapeutic approach as needed.

The agents and compositions of the invention may be administered to humans and other animals for therapy by any suitable route of administration, including orally, nasally, as by, for example, a spray, rectally, intravaginally, parenterally, intracistemally and topically, as by powders, ointments or drops, including buccally and sublingually.

All methods may further include the step of bringing the active ingredient(s) into association with a pharmaceutically acceptable carrier, which constitutes one or more accessory ingredients. Pharmaceutically acceptable carriers useful for formulating an agent for administration to a subject are well known in the art and include, for example, aqueous solutions such as water or physiologically buffered saline or other solvents or vehicles such as glycols, glycerol, oils such as olive oil or injectable organic esters. A pharmaceutically acceptable carrier can contain physiologically acceptable compounds that act, for example, to stabilize or to increase the absorption of the conjugate. Such physiologically acceptable compounds include, for example, carbohydrates, such as glucose, sucrose or dextrans, antioxidants, such as ascorbic acid or glutathione, chelating agents, low molecular weight proteins or other stabilizers or excipients. One skilled in the art would know that the choice of a pharmaceutically acceptable carrier, including a physiologically acceptable compound, depends, for example, on the physico-chemical characteristics of the therapeutic agent and on the route of administration of the composition, which can be, for example, orally or parenterally such as intravenously, and by injection, intubation, or other such method known in the art. The pharmaceutical composition also can contain a second (or more) compound(s) such as a diagnostic reagent, nutritional substance, toxin, or therapeutic agent, for example, a cancer chemotherapeutic agent and/or vitamin(s).

The agents of the invention can be incorporated within an encapsulating material such as into an oil-in-water emulsion, a microemulsion, micelle, mixed micelle, liposome, microsphere or other polymer matrix (see, for example, Gregoriadis, Liposome Technology, Vol. 1 (CRC Press, Boca Raton, Fla. 1984); Fraley, et al., Trends Biochem. Sci., 6:77 (1981), each of which is incorporated herein by reference). Liposomes, for example, which consist of phospholipids or other lipids, are nontoxic, physiologically acceptable and metabolizable carriers that are relatively simple to make and administer. “Stealth” liposomes (see, for example, U.S. Pat. Nos. 5,882,679; 5,395,619; and 5,225,212, each of which is incorporated herein by reference) are an example of such encapsulating materials particularly useful for preparing a pharmaceutical composition useful for practicing a method of the invention, and other “masked” liposomes similarly can be used, such liposomes extending the time that the therapeutic agent remain in the circulation. Cationic liposomes, for example, also can be modified with specific receptors or ligands (Morishita et al., J. Clin. Invest. 91:2580-2585 (1993), which is incorporated herein by reference). In addition, a polynucleotide agent can be introduced into a cell using, for example, adenovirus-polylysine DNA complexes (see, for example, Michael et al., J. Biol. Chem. 268:6866-6869 (1993), which is incorporated herein by reference). The carriers, in addition to those disclosed above, can include glucose, lactose, mannose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition auxiliary, stabilizing, thickening or coloring agents and perfumes can be used, for example a stabilizing dry agent such as triulose (see, for example, U.S. Pat. No. 5,314,695).

The route of administration of a composition containing the inhibitors of the invention will depend, in part, on the chemical structure of the molecule. Polypeptides and polynucleotides, for example, are not particularly useful when administered orally because they can be degraded in the digestive tract. However, methods for chemically modifying polynucleotides and polypeptides, for example, to render them less susceptible to degradation by endogenous nucleases or proteases, respectively, or more absorbable through the alimentary tract are well known (see, for example, Blondelle et al., Trends Anal. Chem. 14:83-92, 1995; Ecker and Crook, BioTechnology, 13:351-360, 1995). For example, a peptide agent can be prepared using D-amino acids, or can contain one or more domains based on peptidomimetics, which are organic molecules that mimic the structure of peptide domain; or based on a peptoid such as a vinylogous peptoid. Where the inhibitor is a small organic molecule such as a steroidal alkaloid, it can be administered in a form that releases the active agent at the desired position in the body (e.g., the stomach), or by injection into a blood vessel such that the inhibitor circulates to the target cells (e.g., cancer cells).

Exemplary routes of administration include, but are not limited to, orally or parenterally, such as intravenously, intramuscularly, subcutaneously, intraperitoneally, intrarectally, intracisternally or, if appropriate, by passive or facilitated absorption through the skin using, for example, a skin patch or transdermal iontophoresis, respectively. Furthermore, the pharmaceutical composition can be administered by injection, intubation, orally or topically, the latter of which can be passive, for example, by direct application of an ointment, or active, for example, using a nasal spray or inhalant, in which case one component of the composition is an appropriate propellant. As mentioned above, the pharmaceutical composition also can be administered to the site of a tumor, for example, intravenously or intra-arterially into a blood vessel supplying the tumor.

The total amount of a compound or composition to be administered in practicing a method of the invention can be administered to a subject as a single dose, either as a bolus or by infusion over a relatively short period of time, or can be administered using a fractionated treatment protocol, in which multiple doses are administered over a prolonged period of time. One skilled in the art would know that the amount of the inhibitors of ETRB activity, BCL-2A1 activity, and/or PARP-3 activity to treat cancer in a subject depends on many factors including the age and general health of the subject as well as the route of administration and the number of treatments to be administered. In view of these factors, the skilled artisan would adjust the particular dose as necessary. In general, the formulation of the pharmaceutical composition and the routes and frequency of administration are determined, initially, using Phase I and Phase II clinical trials.

In general, a suitable daily dose of a compound or composition of the invention will be that amount of the compound or composition that is the lowest dose effective to produce a therapeutic effect. Such an effective dose will generally depend upon the factors described above. Generally, intravenous, intracerebroventricular and subcutaneous doses of the compounds of this invention for a subject will range from about 0.001 to about 100 mg per kilogram of body weight per day which can be administered in single or multiple doses.

If desired, the effective daily dose of the active compound or composition may be administered as two, three, four, five, six or more sub-doses administered separately at appropriate intervals throughout the day, optionally, in unit dosage forms. There may be a period of no administration followed by another regimen of administration.

It will be understood, however, that the specific dose level and frequency of dosage for any particular subject may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

The methods of the invention can be performed by contacting samples of cells ex vivo, for example, in a culture medium or on a solid support. Alternatively, or in addition, the methods can be performed in vivo, for example, by transplanting a cancer cell sample into a test animal (e.g., a nude mouse), and administering the test agent or composition to the test animal. An advantage of the in vivo assay is that the effectiveness of a test agent can be evaluated in a living animal, thus more closely mimicking the clinical situation. Since in vivo assays generally are more expensive, the can be particularly useful as a secondary screen, following the identification of “lead” agents using an in vitro method.

When practiced as an in vitro assay, the methods can be adapted to a high throughput format, thus allowing the examination of a plurality (i.e., 2, 3, 4, or more) of cell samples and/or test agents, which independently can be the same or different, in parallel. A high throughput format provides numerous advantages, including that test agents can be tested on several samples of cells from a single subject, thus allowing, for example, for the identification of a particularly effective concentration of an agent to be administered to the subject, or for the identification of a particularly effective agent to be administered to the subject. As such, a high throughput format allows for the examination of two, three, four, etc., different test agents, alone or in combination, on the cancer cells of a subject such that the best (most effective) agent or combination of agents can be used for a therapeutic procedure. Further, a high throughput format allows, for example, control samples (positive controls and or negative controls) to be run in parallel with test samples, including, for example, samples of cells known to be effectively treated with an agent being tested.

A high throughput method of the invention can be practiced in any of a variety of ways. For example, different samples of cells obtained from different subjects can be examined, in parallel, with same or different amounts of one or a plurality of test agent(s); or two or more samples of cells obtained from one subject can be examined with same or different amounts of one or a plurality of test agent. In addition, cell samples, which can be of the same or different subjects, can be examined using combinations of test agents and/or known effective agents. Variations of these exemplified formats also can be used to identifying an agent or combination of agents useful for treating cancers.

When performed in a high throughput (or ultra-high throughput) format, the methods can be performed on a solid support (e.g., a microtiter plate, a silicon wafer, or a glass slide), wherein samples to be contacted with a test agent are positioned such that each is delineated from each other (e.g., in wells). Any number of samples (e.g., 96, 1024, 10,000, 100,000, or more) can be examined in parallel using such a method, depending on the particular support used. Where samples are positioned in an array (i.e., a defined pattern), each sample in the array can be defined by its position (e.g., using an x-y axis), thus providing an “address” for each sample. An advantage of using an addressable array format is that the method can be automated, in whole or in part, such that cell samples, reagents, test agents, and the like, can be dispensed to (or removed from) specified positions at desired times, and samples (or aliquots) can be monitored, for example, for ETRB activity, BCL-2A1 activity, PARP-3 activity, HIF-1α activity, VEGF activity, GRAVIN activity, and/or cell viability.

Thus, the methods of the invention are adaptable to a wide variety of assays, including labeled in vitro protein-protein binding assays, electrophoretic mobility shift assays, immunoassays for protein binding, functional assays (phosphorylation assays, etc.) and the like. Of particular interest are screening assays for agents that have a low toxicity for human cells. In one embodiment, the methods are useful for binding assays in which an ETRB or the candidate agent is non-diffusibly bound to an insoluble support as described above. Novel binding agents include specific antibodies, non-natural binding agents identified in screens of chemical libraries, peptide analogs, etc.

The determination of the binding of the candidate agent to the ETRB may be done in a number of ways. For example, the candidate agent is labeled, and binding determined directly. This may be done by attaching all or a portion of the ETRB to a solid support, adding a labeled candidate agent (for example a fluorescent label), washing off excess reagent, and determining whether the label is present on the solid support. Various blocking and washing steps may be utilized as is known in the art. Candidate agents that affect ETRB bioactivity may also identified by screening agents for the ability to either enhance or reduce the activity of BCL-2 μl, PARP-3, HIF-1α, VEGF and/or GRAVIN, as discussed above. The methods include both in vitro screening methods, as are generally outlined above, and in vivo screening of cells for alterations in the activities of BCL-2A1, PARP-3, HIF-1α, VEGF and/or GRAVIN.

By “labeled” herein is meant that the compound is either directly or indirectly labeled with a label which provides a detectable signal, e.g. radioisotope, fluorescers, enzyme, antibodies, particles such as magnetic particles, chemiluminescers, or specific binding molecules, etc. Specific binding molecules include pairs, such as biotin and streptavidin, digoxin and antidigoxin etc. For the specific binding members, the complementary member would normally be labeled with a molecule which provides for detection, in accordance with known procedures, as outlined above. The label can directly or indirectly provide a detectable signal.

Incubations may be performed at any temperature which facilitates optimal activity, typically between 4° and 40° C. Incubation periods are selected for optimum activity, but may also be optimized to facilitate rapid high through put screening. Typically between 0.1 and 1 hour will be sufficient. Excess reagent is generally removed or washed away. The second component is then added, and the presence or absence of the labeled component is followed, to indicate binding.

Positive controls and negative controls may be used in the assays of the invention. Preferably all control and test samples are performed in at least triplicate to obtain statistically significant results. Incubation of all samples is for a time sufficient for the binding of the agent to the protein. Following incubation, all samples are washed free of non-specifically bound material and the amount of bound, generally labeled agent determined. For example, where a radiolabel is employed, the samples may be counted in a scintillation counter to determine the amount of bound compound.

A variety of other reagents may be included in the screening assays. These include reagents like salts, neutral proteins, e.g. albumin, detergents, etc., which may be used to facilitate optimal protein-protein binding and/or reduce non-specific or background interactions. Also reagents that otherwise improve the efficiency of the assay, such as protease inhibitors, nuclease inhibitors, anti-microbial agents, etc., may be used. The mixture of components may be added in any order that provides for the requisite binding.

The measurements can be determined wherein all of the conditions are the same for each measurement, or under various conditions, with or without candidate agents, or at different stages of a disease state such as cancer. For example, a measurement can be determined in a cell or cell population wherein a candidate agent is present and wherein the candidate agent is absent. For another example, the cells may be evaluated in the presence or absence or previous or subsequent exposure of physiological signals, for example hormones, antibodies, peptides, antigens, cytokines, growth factors, action potentials, pharmacological agents including chemotherapeutics, radiation, carcinogenics, or other cells (i.e. cell-cell contacts). In yet another example, the measurements of bioactivity are taken wherein the conditions are the same, and the alterations are between one cell or cell population and another cell or cell population.

By a “population of cells” or “library of cells” herein is meant at least two cells, with at least about 10³ being preferred, at least about 10⁵ being particularly preferred, and at least about 10⁸ to 10⁹ being especially preferred. The population or sample can contain a mixture of different cell types from either primary or secondary cultures although samples containing only a single cell type are preferred, for example, the sample can be from a cell line, particularly cancer cell lines.

The following examples are provided to further illustrate the advantages and features of the present invention, but are not intended to limit the scope of the invention. While they are typical of those that might be used, other procedures, methodologies, or techniques known to those skilled in the art may alternatively be used.

### EXAMPLE 1

### Melanoma Cells Derived from More Advanced Lesions Display Higher Sensitivity to ETRB Inhibition

In the present work, four graded human melanoma cell lines were derived from a primary lesion, and cutaneous and lymph node metastases. The correlation between the progression level and responsiveness to BQ788 in each of the cell lines was tested. The melanoma cells derived from more advanced lesions displayed higher sensitivity to ETRB inhibition, thus providing new insight into the mechanisms that may underlie melanoma cell death as a result of ETRB blockade.

Human melanoma cell lines Me191-1/GG (Primary), Me 190/DA (Cut-met), Me 275/EP (LN-met) were obtained from the Ludwig Institute (Epalinges, Switzerland) and SK-MEL-28 was obtained from the American Tissue Culture Collection (ATCC). All cell lines were cultured in RPMI 1640 medium with Glutamax-I (Gibco) containing 10% FBS (Gibco/BRL), and 100 μg/ml antibiotics (Pen-Strep mix; Gibco/BRL) in a humidified incubator with 5% CO₂ at 37° C. Matrigel Matrix (Becton Dickinson) was used according to the manufacturer's instructions in 8.0 μm pore size cell culture inserts (Falcon) in 24 multiwell plates (Becton Dickinson). 20,000 cells were cultured on top of Matrigel Matrix for two weeks. Inserts were fixed in 10% formalin and the fixed Matrigel embedded in paraffin. Longitudinal sections were stained in hematoxylin and eosin to visualize the cells. After removal of the inserts, MTS solution was added to the well to quantify the cells that migrated through the insert and were attached to the bottom of the well by measuring the OD at 492 nm (Promega). BQ788 and BQ123 (Calbiochem) were used as described (Lahav, et al., An endothelin receptor B antagonist inhibits growth and induces cell death in human melanoma cells in vitro and in vivo, Proc. Natl. Acad. Sci. U.S.A., 96:11496-11500 (1999)).

### EXAMPLE 2

### Immunoblotting

Samples were subjected to SDS-PAGE according to the method as described (Laemmli, Cleavage of structural proteins during the assembly of the head of bacteriophage T4, Nature, 227:680-685 (1970)). Proteins were blotted onto PVDF (Millipore) membranes and the filters were blocked with 5% non-fat dry milk. Immunostaining was preformed with anti-human endothelin receptor B antibody (Assay Designs, Inc.) (4 μg/ml) or anti-human Tubulin (Oncogene Research Products) (1 μg/ml) followed by incubation with a peroxidase conjugated goat anti rabbit secondary antibody (Sigma) (1:5000). Bands of 51 kd and 60 kd for ETRB (Hiraki, et al, Regular immunohistochemical localization of endothelin-1 and endothelin receptor B in normal, hyperplastic and neoplastic human adrenocortical cells, Pathol. Int., 47:117-125 (1997)) and tubulin respectively were detected using a chemiluminescent substrate kit (Interchim) according to the manufacturer's recommendations.

### EXAMPLE 3

### cDNA Microarray Analysis

Cells were cultured in the presence of BQ788 or its solvent (HCO60) for 2 days and then lysed and subjected to RNA extraction using SV Total RNA isolation kit (Promega). Two rounds of RNA amplification were conducted using RiboAmp™ RNA Amplification kit (Arcturus). Labeled cDNA was obtained by reverse transcription of 6 μl amplified RNA and incorporation of Cy3-dCTP (5-Amino-propargyl-2′-dexoycytidine 5′-triphosphate coupled to Cy3 fluorescent dye) and Cy5-dCTP (5-Amino-propargyl-2′-dexoycytidine 5′-triphosphate coupled to Cy5 fluorescent dye) (Amersham Biosciences). Human 10k arrays containing PCR products spotted onto glass slides were obtained from the Lausanne DNA Array Facility (DAF). Hybridization of labeled cDNA to microarrays was preformed for 16 h at 64° C. in a humidified chamber (Corning Costar). Scanning was done in a Scanarray 4000 scanner (Perkin Elmer). Image analysis was preformed using the ScanAlyse program (version 2.5). Data analysis was done using the R package: Statistics for Microarray analysis containing com.braju.sma package (www.r-project.org).

### EXAMPLE 4

### Real-Time RT-PCR

RNA was prepared from the different cell lines in different culture conditions using SV total RNA isolation kit (Promega) according to the manufacturer's protocol. For each sample RNA concentration was determined using the total RNA nano procedure of an Agilent bioanalyzer. A stock of SK-MEL-28 RNA aliquots was prepared containing 10, 20, 50, 100, 200, 500 and 1000 ng/12 μl for use as a standard curve and kept in −80° C. For the experimental unknown samples, a stock of 100 ng/12 μl RNA aliquots from each cell line and condition was prepared and stored at −80° C. For each real-time experiment one series of standard curve and unknown experimental series of aliquots was put on ice for use. Complementary DNA was prepared by adding to each tube 0.5 μl of random hexamers (Promega) and 1 μl of 10 mM dNTP mix (Promega) and incubating at 65° C. for 5 min. 4 μl of 5× M-MLV buffer (Promega), 1 μl of the RNase inhibitor RNasin (Promega) and 1 μl of M-MLV RT RNase H (Promega) were then added and the solution was incubated at 42° C. for 50 min followed by 15 min at 70° C. For real time reactions 15 μl of each cDNA sample were transferred to 3 wells in a 386 well plate. To each well we added 10 μl of TaqMan universal PCR master mix (Applied Biosystems), 1 μl of primers+probe mix (Assays-on-Demand from Applied Biosystems: EDNRB Hs00240747, BCL-2A1 Hs00187845, PARP-3 Hs00154151, VEGF Hs00173626, HIF-1α Hs00153153 and Gravin Hs00374507) and 4 μl of nuclease free water (Promega). Wells containing water instead of cDNA served as negative controls. The samples were subjected to 40 cycles of 15 sec at 95° C. and 1 min at 60° C. A standard curve of Ct versus RNA quantity was established and accordingly the Ct in the unknown samples was correlated to quantity of transcript.

### EXAMPLE 5

### Apoptosis

Cells were plated in 24 wells plate in 500 μl medium and cultured for 5 days in the presence of BQ788 or solvent. A cell death detection ELISA PLUS kit (Roche) designed to reveal histone-180 bp DNA fragment complexes of nucleosomes was used to measure apoptosis. Briefly, cells floating in the supernatants and those attached to the dish were incubated in lysis buffer for 30 min. in room temperature (RT). After spinning for 10 min at 200 g, the DNA content of each sample was determined. 20 μl containing equal amounts of DNA were added to each well (3 wells per condition) coated with anti-histone antibody and incubated with additional 80 μl anti-DNA peroxidase immunogen under gentle shaking conditions for 2 h at RT. After washing and revelation with peroxidase substrate the samples OD were measured at 405 nm against substrate containing wells as blank.

### EXAMPLE 6

### Caspase Inhibitors and Caspase 6 Activity

Caspase 3 inhibitor I (inhibits caspases 3, 6, 7, 8 & 10), cell-permeable (Calbiochem) and Caspase 6 inhibitor II, Cell-Permeable (Calbiochem) were dissolved in DMSO. Cells were cultured in 96 wells and the next day divided into 4 groups: control with solvents only DMSO+HCO60 (the solvent of BQ788 according to Ishikawa, et al., Biochemical and pharmacological profile of a potent and selective endothelin B-receptor antagonist, BQ-788, Proc. Natl. Acad. Sci. U.S.A., 91:4892-4896 (1994); Lahav, et al., An endothelin receptor B antagonist inhibits growth and induces cell death in human melanoma cells in vitro and in vivo, Proc. Natl. Acad. Sci. U.S.A., 96:11496-11500 (1999)), caspase inhibitor+HCO60, BQ788+DMS0 and BQ788+caspase inhibitor. Cell viability was measured using MTS solution in parallel experiments at 5 different time points. Caspase 6 activity was measured using Mch2/Caspase-6 Colorimetric Protease Assay Kit according to the manufacturer's instructions. Briefly, 3×10⁵ SK-MEL-28 cells were plated onto 10 cm culture dishes. On the following day, BQ788 was added to experimental plates and its solvent to controls. 3 days later, cells were lysed and the lysates assayed for protein concentration. Caspase 6 activity was measured in 4 samples containing equal amounts of protein from each condition by spectrophotometric detection of a chromophore following its release from the labeled caspase 6 substrate VEID.

### EXAMPLE 7

### RNA Interference (RNAi)

64 mer oligonucleotides were synthesized containing a target 19 mer sequence specific to EDNRB (see bold characters in the sequence). Comparing this sequence to the GenBank human EST data base gave only similarities to EDNRB even when the comparison was reduced to only 15 mers out of the 19. Additional flanking sequences were added for the creation of a stem loop structure and Bgl-II and Hind-III cloning sites (Brummelkamp, et al., A system for stable expression of short interfering RNAs in mammalian cells, Science, 296:550-553 (2002)).

Forward oligonucleotide:

(SEQ ID NO: 1)

5′GATCCCCGTGCATGCGAAACGGTCCCTTCAAGAGAGGGACCGTTTCGC
ATG CACTTTTTGGAAA 3′.
Reverse oligonucleotide:

(SEQ ID NO: 2)

5′AGCTTTTCCAAAAAGTGCATGCGAAACGGTCCCTCTCTTGAAGGGACC
GTTT CGCATGCACGGG 3′.

The oligonucleotides were annealed, phosphorylated and ligated into Bgl-II and Hind-III sites of the pSuper plasmid (Brummelkamp, et al., A system for stable expression of short interfering RNAs in mammalian cells, Science, 296:550-553 (2002)) containing an H1-RNA promoter (kind gift of Netherlands Cancer Institute, Amsterdam). For each cell line 20 to 40×10³ cells were cultured in each well of 96 well plate containing medium without antibiotics. On the following day the cells were transfected with 0.4 μg plasmid+0.8 μl Lipofectamine 2000 (Invitrogen) in Optimem 1 with Glutamax-I (Gibco). The medium was replaced with a complete medium after 6 h. Viability was measured 24 and 48 h after transfection by adding MTS solution (Promega) and measuring the OD at 492 μm. Parallel cultures in 6 well plates were transfected using the same conditions. 24 and 48 h later cell lysates were prepared and protein concentration was determined for each sample. Immunoblotting was preformed on samples with equal amounts of protein as described.

### EXAMPLE 8

### ETRB Antagonism is Most Effective Against Metastatic Melanoma

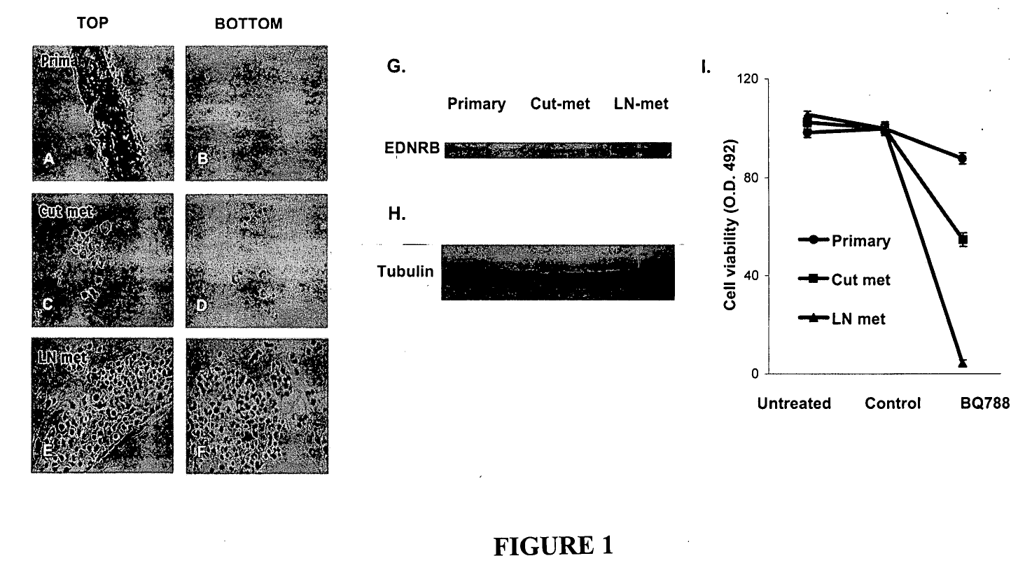

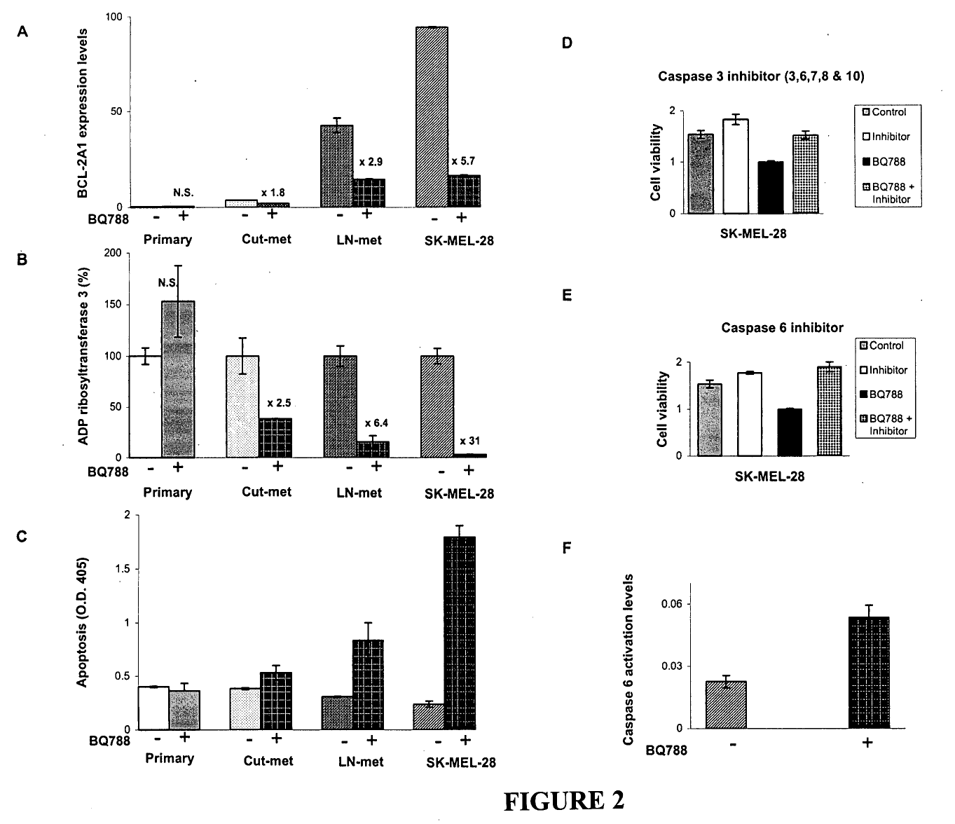

To address the relationship between the level of progression of human melanoma and responsiveness to BQ788 treatment, 3 cell lines that had been grown in culture for a restricted number of passages were used. The cell lines included Me 191-I/GG (Primary), a low passage cell line derived from a primary cutaneous melanoma lesion; Me 190/DA (Cut-met), a cell line derived from a sub-cutaneous metastasis proximal to the primary lesion; and Me 275/EP (LN-met), a cell line derived from a lymph node metastasis of a patient who had subcutaneous metastasis two years earlier. To determine whether the three cell lines retained their phenotype in culture, we assessed their invasiveness in a Matrigel invasion assay. Consistent with the lesions from which they originated, the primary melanoma cells failed to penetrate the gel and remained on its surface (FIG. 1A, B), the sub-cutaneous metastasis-derived cells displayed an intermediate degree of invasiveness (FIG. 1 C, D) and the lymph node-derived cells were highly invasive (FIG. 1 E, F). Western blot analysis showed that expression of ETRB correlated with melanoma cell invasiveness (FIG. 1 G, H). Most importantly, the sensitivity of the cells to BQ788 was proportional to the degree of progression of the tumor from which the cells were derived and the receptor expression level (FIG. 11). Thus, whereas incubation with BQ788 for seven days had little effect on primary melanoma cell viability (12% cell death), it reduced the viability of the Cut-met cells by 45% and that of the LN-met line by 96%. Together with the report that ETRB expression increases in human melanoma as they advance to metastatic disease (Demunter, et al., Expression of the endothelin-B receptor in pigment cell lesions of the skin. Evidence for its role as tumor progression marker in malignant melanoma, Virchows Arch., 438:485-491 (2001)), these observations suggest that ETRB inhibition might be most effective in metastatic melanoma. In addition to inducing morphological changes indicative of differentiation, treatment of the melanoma cells with BQ788 eventually results in reduced cell number and cell death. This effect was quantified using the MTS assay. As shown in FIG. 2, all 7 melanoma lines tested show a very significant loss in the number of viable cells upon treatment with 100 μM BQ788, although some lines are clearly more sensitive than others. In contrast, the number of kidney cells is not reduced by high concentrations of BQ788, demonstrating that this inhibitor is not generally toxic.

### EXAMPLE 9

### ETRB Inhibition Reduces BCL-2A1 and PARP-3 Expression and Induces Apoptosis and Caspase 6 Activation

To explore the possible mechanisms that underlie the loss of viability of metastatic but not primary melanoma cells in response to ETRB antagonism, changes in gene expression profiles of BQ788-treated and untreated cells were investigated by cDNA microarray analysis. In an effort to identify changes that are common to BQ788-sensitive cells we used, in addition to the LN-met cells, the human melanoma cell line SK-MEL-28 that was previously shown to be highly sensitive to BQ788 (Lahav, et al., An endothelin receptor B antagonist inhibits growth and induces cell death in human melanoma cells in vitro and in vivo, Proc. Natl. Acad. Sci. U.S.A., 96:11496-11500 (1999)). Both cell lines were treated with BQ788 and RNA was extracted 2 days after treatment, a time point that precedes significant changes in cell viability by 3-5 days. For each cell line, transcripts from BQ788-treated cells were compared to those derived from control (solvent-treated) cells. Comparing the differentially regulated genes in the two different cell lines resulted in the identification of only a few genes that appeared to be significantly affected by BQ788 at this early time point (Table 1). Two genes that were found to be down-regulated upon treatment with BQ788 are the survival factor BCL-2A1 (Cheng, et al., Upregulation of Bcl-x and Bfl-1 as a potential mechanism of chemoresistance, which can be overcome by NF-kappaB inhibition, Oncogene, 19:4936-4940 (2000); D'Souza, et al., The bfl-1 gene is transcriptionally upregulated by the Epstein-Barr virus LMP 1, and its expression promotes the survival of a Burkitt's lymphoma cell line, J. Virol., 74:6652-6658 (2000); Kenny, et al., GRS, a novel member of the Bcl-2 gene family, is highly expressed in multiple cancer cell lines and in normal leukocytes, Oncogene, 14:997-1001 (1997); Lee, et al., NF-kappaB-mediated up-regulation of Bcl-x and Bfl-1/A1 is required for CD40 survival signaling in B lymphocytes, Proc. Natl. Acad. Sci. U.S.A., 96:9136-9141 (1999); Noble, et al., Monocytes stimulate expression of the Bcl-2 family member, A1, in endothelial cells and confer protection against apoptosis, J. Immunol., 162:1376-1383 (1999); Wang, et al., NF-kappaB induces expression of the Bcl-2 homologue A1/Bfl-1 to preferentially suppress chemotherapy-induced apoptosis, Mol. Cell Biol., 19:5923-5929 (1999); Zong, et al., The prosurvival Bcl-2 homolog Bfl-1/A1 is a direct transcriptional target of NF-kappaB that blocks TNFalpha-induced apoptosis, Genes Dev., 13:382-387 (1999); Pagliari, et al., Macrophages require constitutive NF-kappaB activation to maintain A1 expression and mitochondrial homeostasis, Mol. Cell Biol., 20:8855-8865 (2000); and Pham, et al., Inhibition of constitutive NF-kappa B activation in mantle cell lymphoma B cells leads to induction of cell cycle arrest and apoptosis, J. Immunol., 171:88-95 (2003)) and ADP ribosyltransferase 3 (PARP-3) (Johansson, M., A human poly(ADP-ribose) polymerase gene family (ADPRTL): cDNA cloning of two novel poly(ADP-ribose) polymerase homologues, Genomics, 57:442-445 (1999)). PARP enzymes are activated in response to DNA damage and are implicated in the repair of DNA strand breaks. PARP cleavage, leading to its inactivation and thereby preventing DNA repair and improving endonuclease access to chromatin, is an early event in apoptosis (Oliver, et al., Poly(ADP-ribose) polymerase in the cellular response to DNA damage, apoptosis, and disease, Am. J. Hum. Genet., 64:1282-1288 (1999); Shall, et al., Poly(ADP-ribose) polymerase-1: what have we learned from the deficient mouse model?, Mutat. Res., 460:1-15 (2000)).

If the observed reduction in BCL-2A1 and PARP-3 expression is implicated in ETRB blockade-dependent cell death, one would expect the more resistant cell lines to display less reduction in BCL-2A1 and PARP-3 levels. To test this possibility and to validate our microarray results, we used quantitative real-time PCR. We found that the level of reduction in BCL-2A1 RNA correlates with the reduction in viability of the 4 different cell lines treated with BQ788 (FIG. 2A). The primary melanoma cells displayed low expression of BCL-2A1, which did not change with BQ788 treatment. The Cut-met line displayed slightly higher levels of BCL-2A1, which was reduced 1.8-fold by BQ788. The more sensitive cell lines LN-met and SK-MEL-28 displayed a 2.9- and 5.7-fold decrease in BCL-2A1 levels, respectively, as a result of BQ788 treatment. A similar response pattern was observed for PARP-3 (FIG. 2B).

Because BCL-2A1 and PARP are known regulators of apoptosis (Smulson, et al., Roles of poly(ADP-ribosyl)ation and PARP in apoptosis, DNA repair, genomic stability and functions of p53 and E2F-1, Adv. Enzyme Regul., 40:183-215 (2000); Vander Heiden, et al., Bcl-2 proteins: regulators of apoptosis or of mitochondrial homeostasis?, Nat. Cell Biol., 1:E209-E216, (1999)), we used an apoptosis detection ELISA that detects histone-associated DNA fragments to measure apoptotic cell death in the different cell lines treated with BQ788. The levels of apoptosis were observed to correlate with the levels of reduction in BCL-2A1 and PARP-3 RNA (FIG. 2C). The primary melanoma cells did not show a decrease in BCL-2A1 or PARP-3 levels and did not display apoptosis in response to BQ788 treatment. In contrast, the other cell lines showed a gradual increase in the levels of apoptosis, from a slight increase in the Cut-met line, through an intermediate level in LN-met, to the highest induction in SK-MEL-28.

BCL2-A1 inhibits the activation of caspase 9 but not caspase 3 or 8 in endothelial cells (Duriez, et al., A1 functions at the mitochondria to delay endothelial apoptosis in response to tumor necrosis factor, J. Biol. Chem., 275:18099-18107 (2000)). To verify caspase activity association of ETRB blockade-dependent cell death, we tested the effect of pan caspase inhibitor (caspase 3, 6, 7, 8 and 10) at various time points ranging from 6 h to 7 days in culture on apoptosis triggered by BQ788 in melanoma cells. At the early time points, when the BQ788 effect is still modest, the pan-inhibitor rescued cell viability (FIG. 2D). However, after 5 to 7 days, the pan inhibitor was no longer effective (data not shown). While addressing the possible activation of effector caspases, we observed that at all time points tested, caspase 6 inhibitor was more effective at rescuing cells than the pan-caspase inhibitor (FIG. 2E). After 3 days of treatment with BQ788, caspase 6 activation could be detected (FIG. 2 F). Taken together, these results suggest that ETRB antagonism induces melanoma apoptosis through reduction in BCL-2A1 expression and, at least in part, caspase 6 activation.

### EXAMPLE 10

### ETRB Expression Levels are Important for Melanoma Viability

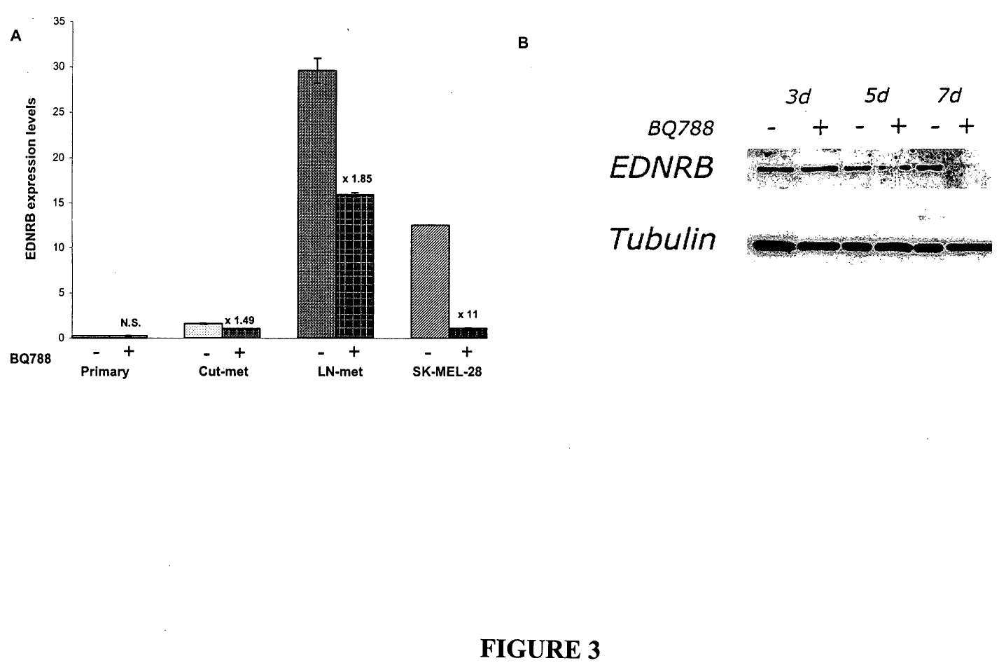

The microarray data suggests that ETRB expression was reduced in melanoma cells treated with BQ788 (Table 1). Further examination of the 4 different melanoma cell lines using realtime RT-PCR confirmed this observation and showed that sensitivity of the melanoma cell lines to BQ788 treatment correlated with the reduction in ETRB mRNA expression (FIG. 3A). The primary melanoma cells did not show any change in ETRB expression levels with BQ788 treatment. The Cut-met line displayed a 1.49-fold reduction, the LN-met line a 1.85-fold decrease and the SK-MEL-28 cells an 11-fold decrease. Western blot analysis showed that ETRB protein levels were reduced upon treatment of SK-MEL-28 cells with BQ788 (FIG. 3B), providing support to the notion that decreased melanoma viability correlates with reduction in ETRB expression.

TABLE 1

| Gene function | Down regulated | Up regulated |
|---|---|---|
| Cell death | BCL2-A1 | |
| | PARP-3 | |
| Angiogenesis | | VEGF |
| Development | Gravin | HIF-1α |
| | EDNRB | |

Table 1. Differentially regulated genes in SK-MEL-28 and LN-met cell lines after 2 days treatment with BQ788 as revealed by microarray analysis and confirmed by real-time RT-PCR.

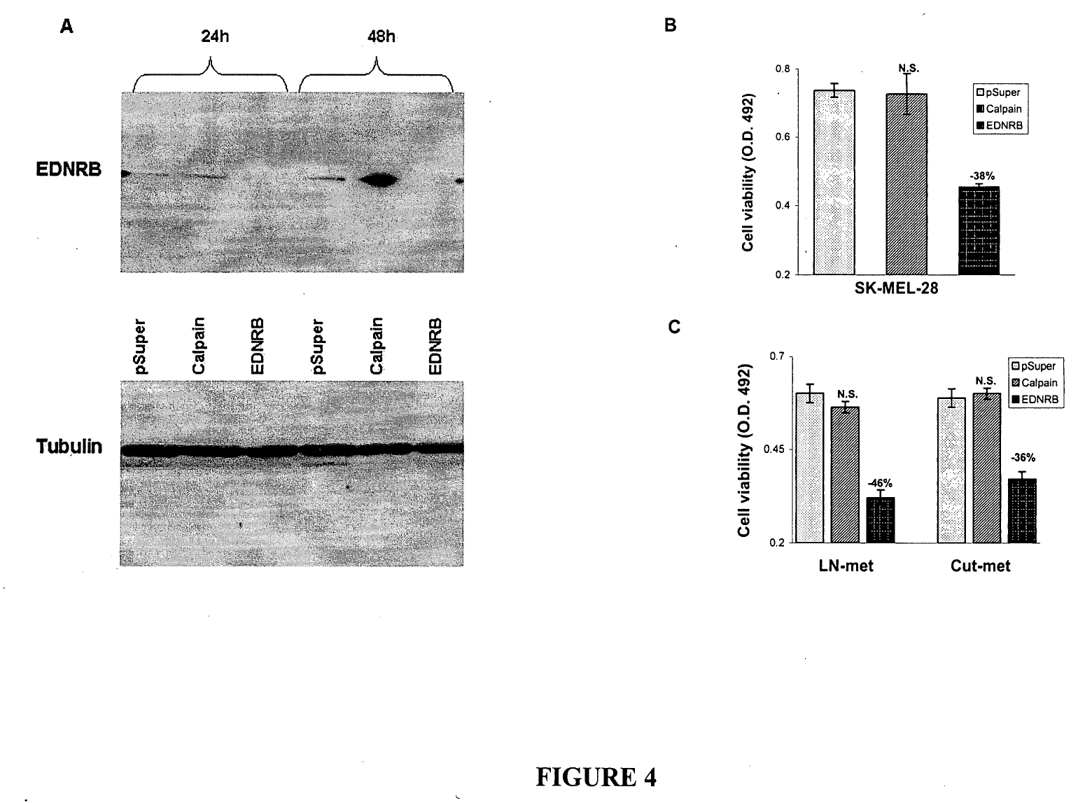

To address the functional relevance of this correlation, small interfering (si)RNA was used to lower the endogenous ETRB levels and found that a reduction in ETRB expression results in reduced melanoma cell viability (FIG. 4). Melanoma cells were transiently transfected with pSuper (Brummelkamp, et al., A system for stable expression of short interfering RNAs in mammalian cells, Science, 296:550-553 (2002)) containing 21 bp fragments that has been shown by BLAST analysis to be ETRB-specific. Despite the fact that only a fraction of the cells were transfected with ETRB siRNA, a significant decrease in ETRB protein levels was observed (FIG. 4) and was accompanied by reduced viability of SK-MEL-28, LN-met and Cut met cells (FIG. 4). These observations are consistent with the notion that ETRB expression may play an important role in the survival of invasive and metastatic melanoma cells.

### EXAMPLE 11

### ETRB Antagonism Leads to Enhanced Angiogenesis

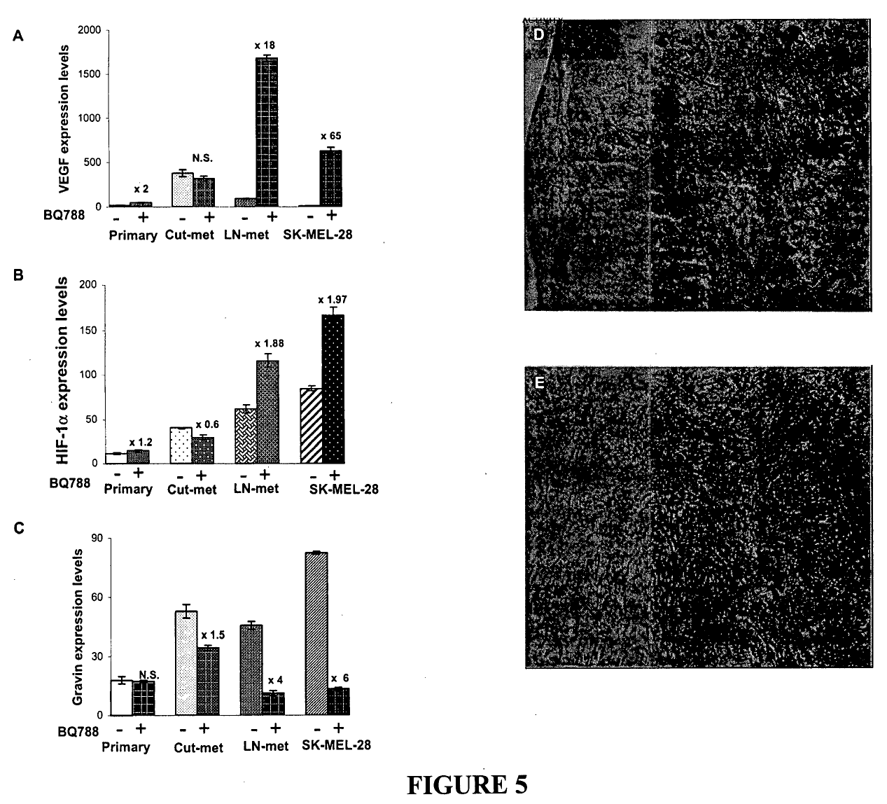

The microarray analysis of BQ788 treated cells also revealed an up-regulation of vascular endothelial growth factor (VEGF) expression. Real-time PCR analysis confirmed that VEGF expression levels increased 18-fold in BQ788 treated LN-met cells and 65-fold in SK-MEL-28 cells, compared to only 2-fold in primary melanoma, and no change in the Cut-met line (FIG. 5). The changes in VEGF expression levels can be explained, at least in part, by changes in the expression levels of its known regulator hypoxia inducible factor-1 oc (HIF-1α) (Maxwell, et al., Oxygen sensors and angiogenesis, Semin. Cell Dev. Biol., 13:29-37 (2002)). HIF-1α RNA expression levels show small but significant changes upon treatment of the cells with BQ788, which correlate in their amplitude with the corresponding changes in VEGF expression. These results suggest that down-regulation of ETRB leads to up-regulation of HIF-1α and VEGF, which is supported by the finding that in ETRB-deficient rats, HIF-1α and VEGF expression levels are higher than normal (Carpenter, et al., Endothelin B receptor deficiency predisposes to pulmonary edema formation via increased lung vascular endothelial cell growth factor expression, Circ. Res., 93:456-463 (2003)). Because VEGF and HIF-1α are regulated by ET-1 via the activation of the endothelin receptor A (ETRA) (Carpenter, et al., Endothelin B receptor deficiency predisposes to pulmonary edema formation via increased lung vascular endothelial cell growth factor expression, Circ. Res., 93:456-463 (2003); Salani, et al., A. Role of endothelin-1 in neovascularization of ovarian carcinoma, Am. J. Pathol., 157:1537-1547 (2000); and Spinella, et al., Endothelin-1 induces vascular endothelial growth factor by increasing hypoxia-inducible factor-1 alpha in ovarian carcinoma cells, J. Biol. Chem., 277:27850-27855 (2002)), we tested the effect of ETRA blockade on the melanoma cell lines used in this study. The ETRA inhibitor BQ123 in combination with BQ788 did not result in changes in VEGF RNA levels compared to those observed when BQ788 was added alone, suggesting that VEGF expression induce by ETRB inhibition is independent of ETRA activity (data not shown).

Interestingly, it was observed that expression of the brain angiogenesis inhibitor, GRAVIN (Lee, et al., SSeCKS regulates angiogenesis and tight junction formation in blood-brain barrier, Nat. Med., 9:900-906 (2003)) is down-regulated in our cell lines upon treatment with BQ788 (FIG. 5C). The relationship between the expression patterns of VEGF and GRAVIN is consistent with that observed in astrocytes, where GRAVIN down-regulates VEGF expression (Lee, et al., SSeCKS regulates angiogenesis and tight junction formation in blood-brain barrier, Nat. Med., 9:900-906 (2003)). In the present study we observed, at least for the most sensitive cell lines, LN-met and SK-MEL-28, that down-regulation of GRAVIN correlates with induction of VEGF. We therefore analyzed tissue sections of tumors derived from a human melanoma cell line grown in nude mice and injected with BQ788 for signs of changes in angiogenesis (Lahav, et al., An endothelin receptor B antagonist inhibits growth and induces cell death in human melanoma cells in vitro and in vivo, Proc. Natl. Acad. Sci. U.S.A., 96:11496-11500 (1999)). Despite inhibition of growth, BQ788-treated tumors showed a clear increase in angiogenesis as revealed by immunohistochemistry staining of vascular endothelial cells with anti-CD31 antibody (FIG. 5). These results strongly suggest that the changes in RNA levels of regulators of angiogenesis induced by ETRB inhibition, particularly that of VEGF expression, correlate with increased angiogenesis in vivo.

### EXAMPLE 12

### ETRB, BCL-2A1 and VEGF Expression in Human Melanoma Samples

To determine if the observed changes in gene expression reflect the situation in human melanomas in vivo, the data of Bittner et al., Molecular classification of cutaneous malignant melanoma by gene expression profiling, Nature, 406:536-540 (2000), who carried out microarray analysis on 31 different samples of cutaneous melanoma and showed ETRB to be generally highly expressed was referred to. The data regarding the expression levels of ETRB, BCL-2A1, VEGF and GAPDH from the different melanoma samples was then analyzed. For ETRB and GAPDH there were two sets of expression data as shown in Table 2. The samples were sorted according to increasing levels of ETRB expression (i.e., according to column B in Table 2).

The melanoma samples were then divided into two groups. Group I included the half of the samples with lower levels of ETRB (mean value of 2.32) and group II those with higher expression levels (mean value of 16.7). It was then determined whether the lower levels of ETRB expression correlate with reduced BCL-2A1 and increased VEGF. Statistical analysis indicates that the second set of data for ETRB in column C also shows low expression levels in group I (mean 3.22) and high expression in group II (mean 8.1,p<0.05). For BCL2-A1 it was determined, as expected, that human melanoma samples with low levels of ETRB expression in group I show significantly lower expression levels of BCL-2A1 (mean 1.3) than their group II counterparts (mean 2.9, p<0.05). For VEGF, a similar relationship to that which was observed in the experiments, namely that lower levels of ETRB expression correlate with significantly higher levels of VEGF transcripts, mean 2.81 compared to 0.9 in group II, p>0.05. There was no difference in GAPDH expression levels between the two melanoma sample groups. This analysis suggests that the relationship between the levels of gene expression in our experimental system probably reflects the situation in human melanomas in general where expression of ETRB and BCL-2A1 tend to behave similarly and inversely correlate with that of VEGF. It was therefore predicted that inhibiting ETRB would result in ETRB and BCL-2A1 down-regulation and VEGF up-regulation in most human melanomas at advanced stages of progression.

As shown in Table 2, expression levels of ETRB (column B and C), BCL-2A1 (D), VEGF (E) and GAPDH (F and G) in 31 human melanoma samples (A) (Bittner et al, 2000) were sorted according to increasing levels of ETRB expression (B) starting from the lowest levels (top) to the highest (bottom). The list was separated into two groups. Group I (top) with lower ETRB expression and Group II with higher ETRB expression levels. Mean values for each group and standard errors (SE) were calculated for each gene. P values and fold change in expression levels of each gene between the two groups are indicated at the bottom.

It was shown that blocking ETRB with BQ788 can induce melanoma cell death in culture and that melanoma sensitivity to ETRB inhibition increases with tumor progression. Cell death induced by inhibition of ETRB was preceded by the repression of the ETRB, BCL-2A1, PARP-3 and GRAVIN genes and induction of the VEGF gene, providing new insight into possible mechanisms that underlie ETRB involvement in the control of cell survival and its potential use as a therapeutic target.

TABLE 2

| A | B | C | D | E | F | G |
|---|---|---|---|---|---|---|
| Mel Sample | EDNRB | EDNRB | BCL2A | VEGF | GAPDH | GAPDH |
| UACC-2873 | 0.69 | 1.23 | 0.69 | 2.67 | 0.87 | 0.86 |
| UACC-930 | 0.71 | 1.43 | 1.36 | 3.13 | 0.89 | 0.89 |
| UACC-1012 | 0.78 | 1.06 | 0.93 | 0.14 | 1.17 | 1.08 |
| UACC-827 | 0.93 | 2.48 | 1.23 | 3.85 | 1.24 | 1.69 |
| M93-47 | 1.06 | 1.73 | 0.95 | 3.94 | 0.9 | 0.9 |
| WM1791-C | 1.27 | 1.85 | 1.09 | 11.29 | 0.95 | 1.03 |
| UACC-1097 | 1.73 | 2.4 | 1.24 | 0.75 | 0.89 | 0.88 |
| UACC-091 | 1.89 | 5.53 | 1.36 | 1.44 | 1.13 | 1 |
| UACC-903 | 2.16 | 2.34 | 1.6 | 4.15 | 1.01 | 0.91 |
| UACC-647 | 2.54 | 3.98 | 1.37 | 4.4 | 0.9 | 0.88 |
| M93-007 | 3.38 | 4.22 | 1.32 | 1.65 | 1.2 | 1.21 |
| UACC-1273 | 3.47 | 5.31 | 1.3 | 0.44 | 1.26 | 1.4 |
| UACC-502 | 4.6 | 5.6 | 1.25 | 1.07 | 0.74 | 0.66 |
| HA-A | 4.78 | 4.7 | 2.25 | 1.06 | 0.71 | 0.8 |
| M92-001 | 4.81 | 4.51 | 1.7 | 2.21 | 1.19 | 1.26 |
| Statistics | | | | | | |
| Mean Group | 2.32 | 3.22 | 1.3 | 2.81 | 1 | 1.03 |
| SE | 1.288 | 1.5 | 0.2 | 1.85 | 0.15 | 0.19 |
| M91-054 | 5.19 | 7.32 | 1.08 | 0.81 | 1.27 | 1.03 |
| UACC-383 | 5.34 | 8.93 | 2.8 | 0.71 | 1.32 | 1.11 |
| TC-F027 | 6.33 | 3.76 | 1.95 | 0.09 | 1 | 0.92 |
| UACC-1529 | 7.91 | 6.38 | 1.92 | 1.33 | 1.11 | 0.96 |
| A-375 | 8.09 | 9.31 | 1.47 | 0.25 | 1.48 | 1.47 |
| TD-1384 | 8.38 | 3.88 | 2.14 | 0.99 | 0.94 | 0.96 |
| UACC-457 | 10.25 | 6.45 | 2.84 | 0.26 | 0.78 | 0.78 |
| UACC-1256 | 11.16 | 7.92 | 1.43 | 1.47 | 1.54 | 1.52 |
| UACC-2534 | 13.6 | 13.12 | 1.89 | 0.45 | 0.99 | 1.22 |
| UACC-3093 | 14.2 | 4.7 | 2.89 | 0.68 | 0.84 | 0.85 |
| TC-1376-3 | 16.83 | 9.09 | 4.53 | 1.37 | 0.89 | 0.83 |
| TD-1638 | 17.93 | 9.94 | 1.77 | 0.88 | 0.98 | 0.91 |
| TD-1376-3 | 20.88 | 12.9 | 9.85 | 1.43 | 0.8 | 0.87 |
| TD-1730 | 25.03 | 13.36 | 2.72 | 2.66 | 1.18 | 1.11 |
| TD-1720 | 29.1 | 6.39 | 6.08 | 1.07 | 1.08 | 1.12 |
| UACC-1022 | 67.19 | 6.17 | 1.93 | 0.81 | 1.43 | 1.29 |
| Statistics | | | | | | |
| Mean Group II | 16.7 | 8.1 | 2.9 | 0.9 | 1.1 | 1 |
| SE | 9.5 | 2.4 | 1.4 | 0.4 | 0.2 | 0.1 |
| p | 0.001 | 9.08E−06 | 0.008 | 0.01 | 0.21 | 0.73 |
| Fold change | 7.2 | 2.5 | 2.2 | −2.9 | N.S. | N.S. |

ETRB expression has been shown to increase with melanoma progression, being highest in metastatic lesions (Demunter, et al., Expression of the endothelin-B receptor in pigment cell lesions of the skin. Evidence for its role as tumor progression marker in malignant melanoma, Virchows Arch., 438:485-491 (2001)). Treatment with BQ788 resulted in a decrease in ETRB expression in high grade melanomas, and sensitivity to the drug correlated with the degree of reduction in ETRB expression: the greater the decrease in ETRB expression, the greater the induction of apoptosis in the treated melanoma cells. Repression of ETRB using siRNA decreased melanoma cell viability, suggesting that high grade melanoma cells depend, at least in part, on ETRB-derived signals for their survival.

Clues as to the mechanisms whereby ETRB promotes melanoma cell survival may be found in the striking correlation between ETRB expression and that of the survival factor BCL-2A1 and the DNA repair factor PARP-3. Not only were the higher expression levels of ETRB in high grade melanomas accompanied by elevated BCL-2A1 and PARP-3 expression, but the decrease in ETRB expression in response to BQ788 was paralleled by a corresponding reduction in the expression of both BCL-2A1 and PARP-3. BCL-2A1 protects several cell types from apoptosis, including monocytes, macrophages, endothelial cells, neutrophils, and B cell lymphomas (D'Souza, et al., The bfl-1 gene is transcriptionally upregulated by the Epstein-Barr virus LMP 1, and its expression promotes the survival of a Burkitt's lymphoma cell line, J. Virol., 74:6652-6658 (2000); Lee, et al., NF-kappaB-mediated up-regulation of Bcl-x and Bfl-1/A1 is required for CD40 survival signaling in B lymphocytes, Proc. Natl. Acad. Sci. U.S.A., 96:9136-9141 (1999); Noble, et al., Monocytes stimulate expression of the Bcl-2 family member, A1, in endothelial cells and confer protection against apoptosis, J. Immunol., 162:1376-1383 (1999); Pagliari, et al., Macrophages require constitutive NF-kappaB activation to maintain A1 expression and mitochondrial homeostasis, Mol. Cell Biol., 20:8855-8865 (2000); and Pham, et al., Inhibition of constitutive NF-kappa B activation in mantle cell lymphoma B cells leads to induction of cell cycle arrest and apoptosis, J. Immunol., 171:88-95 (2003)), and mediates chemo-resistance in some human cancer cell lines (Cheng, et al., Upregulation of Bcl-x and Bfl-1 as a potential mechanism of chemoresistance, which can be overcome by NF-kappaB inhibition, Oncogene, 19:4936-4940 (2000); Wang, et al., NF-kappaB induces expression of the Bcl-2 homologue A1/Bfl-1 to preferentially suppress chemotherapy-induced apoptosis, Mol. Cell Biol., 19:5923-5929 (1999); and Zong, et al., The prosurvival Bcl-2 homolog Bfl-1/A1 is a direct transcriptional target of NF-kappaB that blocks TNFalpha-induced apoptosis, Genes Dev., 13:382-387 (1999)). PARP-3 belongs to a family of constitutive factors of the DNA damage surveillance network (Augustin, et al., PARP-3 localizes preferentially to the daughter centriole and interferes with the G1/S cell cycle progression, J. Cell Sci., 116:1551-1562 (2003)). PARP-1 promotes transcriptional activation of NFKB (Hassa, et al., A role of poly (ADP-ribose) polymerase in NF-kappaB transcriptional activation, Biol. Chem., 380:953-959 (1999); Oliver, et al., Poly(ADP-ribose) polymerase in the cellular response to DNA damage, apoptosis, and disease, Am. J. Hum. Genet., 64:1282-1288 (1999)), a known inducer of BCL-2A1 (Cheng, et al., Upregulation of Bcl-x and Bfl-1 as a potential mechanism of chemoresistance, which can be overcome by NF-kappaB inhibition, Oncogene, 19:4936-4940 (2000); Lee, et al., NF-kappaB-mediated up-regulation of Bcl-x and Bfl-1/A1 is required for CD40 survival signaling in B lymphocytes, Proc. Natl. Acad. Sci. U.S.A., 96:9136-9141 (1999); Pagliari, et al., Macrophages require constitutive NF-kappaB activation to maintain A1 expression and mitochondrial homeostasis, Mol. Cell Biol., 20:8855-8865 (2000); Pham, et al., Inhibition of constitutive NF-kappa B activation in mantle cell lymphoma B cells leads to induction of cell cycle arrest and apoptosis, J. Immunol., 171:88-95 (2003); and Zong, et al., The prosurvival Bcl-2 homolog Bfl-1/A1 is a direct transcriptional target of NF-kappaB that blocks TNFalpha-induced apoptosis, Genes Dev., 13:382-387 (1999)). It seems likely that ETRB inhibition, leading to down-regulation of PARP-3 and BCL-2A1, reduces DNA repair and apoptosis resistance mechanisms. Consistent with this notion, repression of BCL-2A1 and PARP-3 was followed by apoptosis.

Down-regulation of BCL2-A1 results in caspase 9 activation but not caspase 3 or 8 cleavage (Duriez, et al., A1 functions at the mitochondria to delay endothelial apoptosis in response to tumor necrosis factor, J. Biol. Chem., 275:18099-18107 (2000); Pagliari, et al., Macrophages require constitutive NF-kappaB activation to maintain A1 expression and mitochondrial homeostasis, Mol. Cell Biol., 20:8855-8865 (2000)). Western blot analysis did not reveal caspase 3 activation (data not shown) but a colorimetric assay detected caspase 6-like activity and caspase 6 inhibitors rescued cells from BQ788-induced apoptosis. These observations suggest that caspase 6 may play an important role in the induction of apoptosis mediated by ETRB inhibition. Whether caspase 6 activation occurs via reduction in BCL-2A1 expression or by alternative pathways remains to be determined.

Interestingly, it was observed that repression of genes implicated in protection against apoptosis by BQ788-mediated inhibition of ETRB was accompanied by the induction of VEGF expression. Accordingly, the growth inhibitory effect of BQ788 on human melanoma xenografts was associated with increased angiogenesis. Consistent with these observations, ETRB deficient rats display basal VEGF expression levels that are higher than in wild type rats (Carpenter, et al., Endothelin B receptor deficiency predisposes to pulmonary edema formation via increased lung vascular endothelial cell growth factor expression, Circ. Res., 93:456-463 (2003). Examination of expression data from 31 human melanomas (Bittner, et al., Molecular classification of cutaneous malignant melanoma by gene expression profiling, Nature, 406:536-540 (2000)) further shows that tumors with relatively low ETRB expression have lower BCL2-A1 and higher VEGF expression levels than tumors with high ETRB expression.

Finally, GRAVIN, also known as A-kinase anchoring protein (AKAP), that serves as a scaffold to coordinate the location of protein kinase A (PKA) and protein kinase C (PKC), was observed to be repressed in response to ETRB inhibition. The functional significance of the decrease in GRAVIN expression is unclear. However, subcellular localization of signaling enzymes plays a central role in the control of cellular events. Correct intracellular targeting of kinases and phospahatases to their preferred substrates is essential to reduce indiscriminate phosphorylation and dephosphorylation that could alter the activation and function of vital cellular mechanisms and potentially compromise cell survival itself. Reduction of expression of scaffold molecules such as GRAVIN may conceivably intefere with important events in cell physiology and potentially contribute to reduced cell viability.

Taken together, these observations suggest that ETRB inhibition provides a promising new therapeutic avenue for the control of invasive and metastatic melanoma. Currently, there is no effective cure for metastatic melanoma, and although various approaches are being implemented, an efficient way to eliminate or even reduce metastatic lesions has yet to be developed.

The observation that ETRB inhibition induces VEGF, which plays a key role in angiogenesis could be important for the design of clinical trials. VEGF induces angiogenesis, which promotes tumor growth and invasiveness, but also increases vascular permeability that could potentially enhance drug delivery (Lejeune, F. J., Clinical use of TNF revisited: improving penetration of anti-cancer agents by increasing vascular permeability, J. Clin. Invest, 110:433-435 (2002)). BQ788 administration results in inhibition of tumors grown in nude mice and leads to shrinkage of tumors treated systemically. By stimulating angiogenesis and vascular permeability, BQ788 action may help create conditions that enhance tumor cell accessibility and thereby amplify its own pro-apoptotic action. Thus, BQ788 may be of value in combination with other drugs whose delivery it may facilitate by promoting angiogenesis. BQ788 has already been assessed in clinical trials for hypertension in patients and healthy volunteers and was not found to be toxic (Strachan, et al., Systemic blockade of the endothelin-B receptor increases peripheral vascular resistance in healthy men, Hypertension, 33:581-585 (1999); and Cardillo, et al., Role of endothelin in the increased vascular tone of patients with essential hypertension, Hypertension, 33:753-758 (1999)).

Endothelin receptor B (ETRB) is overexpressed in most human melanomas and is proposed to provide a marker of melanoma progression. It has been shown previously that inhibition of ETRB leads to increased human melanoma cell death in vitro and in vivo, resulting in shrinkage of tumors grown in immunocompromised mice (see U.S. Pat. No. 6,545,048, which is incorporated herein by reference). In the present work the effects of ETRB inhibition on 4 human melanoma cell lines derived from tumors at distinct stages of progression was analyzed. The data collected indicates that the ETRB antagonist BQ788 induces apoptosis most effectively in metastatic melanoma cells. Microarray analysis shows that BQ788 treatment leads to a reduction in the expression of the survival factor BCL-2A1 and the DNA repair factor poly(ADP-ribose) polymerase 3 (PARP-3) that is more pronounced in cells derived from metastatic than primary melanoma. Decreased cell viability was observed to correlate with reduction in ETRB expression, and reduction in ETRB protein levels by siRNA led to an increase in cell death. Interestingly, reduction of ETRB expression by BQ788 was accompanied by a strong induction of VEGF expression and repression of the angiogenic suppressor GRAVIN. These changes in gene expression correlated with increased angiogenesis in tumors injected with ETRB antagonist in vivo. Moreover, analysis of gene expression data from human melanomas shows a general tendency of ETRB over-expression to be accompanied by up-regulation of BCL-2A1 and down-regulation of VEGF, suggesting that the observations provided by the present study are relevant to human disease.

Although the invention has been described with reference to the above examples, it will be understood that modifications and variations are encompassed within the spirit and scope of the invention. Accordingly, the invention is limited only by the following claims.

What is claimed is:

1. A method of treating cancer comprising administering to a subject in need thereof, a therapeutically effective amount of a nucleic acid molecule that results in silencing endothelin receptor B activity through RNAi in cancer cells of the subject.

2. The method of claim 1, wherein the administering is systemic or parenteral.

3. The method of claim 1, wherein the administering is directly to a tumor site.

4. The method of claim 1, wherein the cancer is cardiac, lung, gastrointestinal, genitourinary tract, liver, bone, nervous system, gynecological, hematologic, skin, or adrenal glands.

5. The method of claim 1, wherein the cancer is metastasizing.

6. The method of claim 1, wherein the cancer is malignant.

7. A method of treating melanoma comprising administering to a subject in need therof, a therapeutically effective amount of a selective inhibitor of BCL-2A1 activity.

8. The method of claim 7, wherein the administering is systemic or parenteral.

9. The method of claim 8, wherein the administering is directly to a tumor site.

10. The method of claim 7, wherein the melanoma is cutaneous or lymph node metastases.

11. The method of claim 7, wherein the inhibitor is a phthalazin-1 (2H)-one, a isoindolinone, nicotinamide, 3-aminobenzamide, benzamide, 4-amino-1,8-napthalimide, 6(5H)-Phenanthridinone, 5-aminoisoquinolinone hydrochloride, 4-hydroxyquinazoline, 4-quinazolinol, 1,5-isoquinolinediol, or 5-hydroxy-1(2H)-isoquinolinone, and 3,4-dihydro-5-[4-(1-piperidinyl)butoxy]-1(2H)-isoquinolinone.

12. A method of treating melanoma comprising administering to a subject in need thereof, a therapeutically effective amount of a selective inhibitor of PARP-3 activity.

13. The method of claim 12, wherein the administering is systemic or parenteral.

14. The method of claim 12, wherein the administering is directly to a tumor site.

15. The method of claim 12, wherein the melanoma is cutaneous or lymph node metastases.

16. The method of claim 12, wherein the inhibitor is a reticulon proteins, sodium butyrate, or antimycin A, ethyl 2-amino-6-bromo-4-[1-cyano-2-ethoxy-2-oxoethyl]-4H4chromene-3-carboxylate (HA14-1).

17. A method of treating cancer comprising administering to a subject in need of thereof, a therapeutically effective amount of a selective inhibitor of an endothelin receptor B activity in combination with a therapeutically effective amount of a therapeutic agent.

18. The method of claim 17, wherein the administering is systemic or parenteral.

19. The method of claim 17, wherein the administering is directly to a tumor site.

20. The method of claim 17, wherein the cancer is cardiac, lung, gastrointestinal, genitourinary tract, liver, bone, nervous system, gynecological, hematologic, skin, or adrenal glands.

21. The method of claim 17, wherein the cancer is metastasizing.

22. The method of claim 17, wherein the cancer is malignant.

23. The method of claim 17, wherein the inhibitor is BQ788 or a derivative thereof.

24. The method of claim 17, wherein the inhibitor is an antisense molecule to an endothelin receptor-B nucleic acid, an endothelin receptor-B agonist nucleic acid, or siRNA.

25. The method of claim 17, wherein the therapeutic agent is an antiangiogenic agent.

26. The method of claim 25, wherein the antiangiogenic agent is thalidomide, rofecoxib, celecoxib, bevacizumab, angiostatin, or endostatin.

27. The method of claim 17, wherein the therapeutic agent is a chemotherapeutic agent.

28. The method of claim 27, wherein the chemotherapeutic agent is an antimetabolite, a DNA cross-linking agent, an alkylating agent, a topoisomerase I inhibitor, or a microtubule inhibitor.

29. A method of identifying cancer cells amenable to treatment with a nucleic acid molecule that results in silencing endothelin receptor B activity through RNAi, comprising detecting elevated endothelin receptor B activity in a sample of cells as compared to endothelin receptor B activity in corresponding normal cells, thereby identifying cancer cells amenable to treatment with a nucleic acid molecule that results in silencing endothelin receptor B activity through RNAi.

30. The method of claim 29, wherein the cells are from a biopsy sample obtained from a subject.

31. The method of claim 29, wherein the cells are from a bodily fluid obtained from a subject.

32. The method of claim 29, further comprising contacting the cells with a nucleic acid molecule that results in silencing endothelin receptor B activity through RNAi in the cells, and detecting a decrease in endothelin B receptor activity following said contact, thereby confirming that the cancer cells are amenable to treatment with a nucleic acid molecule that results in silencing endothelin receptor B activity through RNAi.

33. A method of identifying melanoma cells amenable to treatment with a selective inhibitor of BCL-2A1 activity or PARP-3 activity, comprising detecting elevated endothelin receptor B activity in a sample of cells as compared to endothelin receptor B activity in corresponding normal cells, thereby identifying melanoma cells amenable to treatment with a selective inhibitor of BCL-2A1 activity or PARP-3 activity.

34. The method of claim 33, wherein the cells are from a biopsy sample obtained from a subject.

35. The method of claim 33, wherein the cells are from a bodily fluid obtained from a subject.

36. The method of claim 33, further comprising contacting the cells with a selective inhibitor of BCL-2A1 activity or PARP-3 activity and detecting a decrease in endothelin B receptor activity following said contact, thereby confirming that the melanoma cells are amenable to treatment with a selective inhibitor of BCL-2A1 activity or PARP-3 activity.

37. A method of identifying cancer cells of a subject amenable to treatment with a selective inhibitor of an endothelin receptor B activity in combination with a therapeutic agent, comprising contacting a sample of cancer cells of a subject with a selective inhibitor of an endothelin receptor B activity and detecting an increase in angiogenesis in the cells as compared to the level of angiogenesis in corresponding normal cells or untreated cancer cells, thereby identifying cancer cells of a subject amenable to treatment with a selective inhibitor of an endothelin receptor B activity in combination with a therapeutic agent.

38. The method of claim 37, comprising detecting elevated levels of HIF-1α activity in the sample of cells from the subject as compared to HIF-1α activity in corresponding normal cells or untreated cancer cells.

39. The method of claim 37, comprising detecting elevated levels of VEGF activity in the sample of cells from the subject as compared to VEGF activity in corresponding normal cells or untreated cancer cells.

40. The method of claim 37, comprising detecting decreased levels of GRAVIN activity in the sample of cells from the subject as compared to GRAVIN activity in corresponding normal cells or untreated cancer cells.

41. The method of claim 37, wherein the therapeutic agent is an antiangiogenic agent.

42. The method of claim 41, wherein the antiangiogenic agent is thalidomide, rofecoxib, celecoxib, bevacizumab, angiostatin, or endostatin.

43. The method of claim 37, wherein the therapeutic agent is a chemotherapeutic agent.

44. The method of claim 43, wherein the chemotherapeutic agent is an antimetabolite, a DNA cross-linking agent, an alkylating agent, a topoisomerase I inhibitor, or a microtubule inhibitor.

45. The method of claim 37, wherein the cells are from a biopsy sample obtained from a subject.

46. The method of claim 37, wherein the cells are from a bodily fluid obtained from a subject.

47. The method of claim 37, further comprising contacting the cells with a therapeutic agent and detecting inhibited growth, enhanced cell death or apoptosis following said contact, thereby confirming that the cancer cells are amenable to treatment with a selective inhibitor of an endothelin receptor B activity in combination with a therapeutic agent.

48. The method of claim 47, wherein the therapeutic agent is an antiangiogenic agent.

49. The method of claim 48, wherein the antiangiogenic agent is thalidomide, rofecoxib, celecoxib, bevacizumab, angiostatin, or endostatin.

50. The method of claim 47, wherein the therapeutic agent is a chemotherapeutic agent.

51. The method of claim 50, wherein the chemotherapeutic agent is an antimetabolite, a DNA cross-linking agent, an alkylating agent, a topoisomerase I inhibitor, or a microtubule inhibitor.

52. A method of identifying an agent useful for treating cancer in combination with a selective inhibitor of an endothelin receptor B activity, comprising contacting a sample of cancer cells with at least one test agent in combination with a selective inhibitor of an endothelin receptor B activity, wherein detection of apoptosis following said contact identifies the agent as useful for treating cancer.

53. The method of claim 52, which is performed in a high throughput format.

54. The method of claim 53, comprising contacting samples of cancer cells of a plurality of samples with at least one test agent in combination with a selective inhibitor of an endothelin receptor B activity.

55. The method of claim 53, wherein the plurality of samples are obtained form a single subject.

56. The method of claim 53, wherein the plurality of samples are obtained from different subjects.

57. A method for monitoring a therapeutic regimen for treating a subject having melanoma, comprising determining a change in BCL-2A1 activity during therapy.

58. The method of claim 57, wherein the therapy comprises the treatment of claim 7.

59. A method for monitoring a therapeutic regimen for treating a subject having melanoma, comprising determining a change in PARP-3 activity during therapy.

60. The method of claim 59, wherein the therapy comprises the treatment of claim 12.

61. A method for monitoring a therapeutic regimen for treating a subject having cancer, comprising determining a change in HIF-1α, VEGF, or GRAVIN activity during therapy.

62. The method of claim 61, wherein the therapy comprises the treatment of claim 1.

63. The method of claim 62, wherein the therapy comprises the treatment of claim 17.


SEQUENCE LISTING (2 sequences)

SEQ ID NO: 1 - DNA, 64 bp, Artificial sequence
  Synthetic construct Forward oligonucleotide
gatccccgtgcatgcgaaacggtcccttcaagagagggaccgtttcgcatgcactttttggaaa

SEQ ID NO: 2 - DNA, 64 bp, Artificial sequence
  Synthetic construct Reverse oligonucleotide
agcttttccaaaaagtgcatgcgaaacggtccctctcttgaagggaccgtttcgcatgcacggg